SEQUENCE LISTING (9 sequences)

SEQ ID NO: 1 - DNA, 11 bp, Unknown
  A hemi-methylated cleaved by BglI
gccnnnnnggc

SEQ ID NO: 2 - DNA, 23 bp, Artificial sequence
  Primer
gcaccgcctggagaagacctacg

SEQ ID NO: 3 - DNA, 19 bp, Artificial sequence
  Primer
ggcggggctcaggatgtcg

SEQ ID NO: 4 - DNA, 21 bp, Artificial sequence
  Nested primer
gagcagcacgagaccgccatc

SEQ ID NO: 5 - DNA, 21 bp, Artificial sequence
  Nested primer
gttgttcaccacgccctccag

SEQ ID NO: 6 - DNA, 21 bp, Artificial sequence
  Primer
gcctcttcgctattacgccag

SEQ ID NO: 7 - DNA, 22 bp, Artificial sequence
  Primer
cggctcgtatgttgtgtggaat

SEQ ID NO: 8 - DNA, 31 bp, Artificial sequence
  Oligonucleotide strand, wherein N denotes a random nucleotide
gggaataagggcgacacggaaatggtaccnn

SEQ ID NO: 9 - DNA, 29 bp, Artificial sequence
  Oligonucleotide strand
ggtaccatttccgtgtcgcccttattccc

(19) United States
(12) Patent Application Publication
(10) Pub. No.: US 2004/0191873 A1
(43) Pub. Date: Sep. 30, 2004

(54) Method for high throughput elucidation of transcriptional profiles and genome annotation

(76) Inventors: Won-Bin Young, Ames, IA (US); Charles J. Link JR., Des Moines, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/810,976

(22) Filed: Mar. 26, 2004

Related U.S. Application Data
(60) Provisional application No. 60/458,152, filed on Mar. 27, 2003.

(51) Int. Cl. C12Q 1/68; C12P 19/34; C12N 15/85

(52) U.S. Cl. 435/91.2; 435/455

(57) ABSTRACT

The invention relates to methods for the elucidation of a transcriptional profile of a cell and for methods to facilitate functional genome annotation. The method of this invention relies on random integration of an artificial exon or a nucleotide fragment that tags the RNA of the gene into which it integrates. In eukaryotic cells, it allows the simultaneous identification of exon-intron boundaries for genome annotation. The gene trapping based strategy for transcript tagging is followed by recovery of those tags fused to flanking cellular exon sequences. Polymerization of those tags allows for high-throughput sequencing and characterization of exon-intron boundaries. Moreover, determination of the frequencies of each particular tag is used to determine the relative transcriptional level of each tagged RNA variant.

### CROSS-REFERENCE TO RELATED APPLICATIONS

This application claims priority to Provisional Application Serial No. 60/458,152, filed Mar. 27, 2003, herein incorporated by reference in its entirety.

### FIELD OF THE INVENTION

This invention relates generally to the field of functional genomics and transcriptomics. The invention enables the elucidation of a transcription profile for a cell with the simultaneous identification of boundaries between exons of genes encoding the proteins contained within the cell.

### BACKGROUND OF THE INVENTION

International Application No. PCT/IUSO/08770 (International Publication WO 01/70948), herein incorporated by reference, discloses methods for elucidating a protein profile for a given cell. One such method comprises inserting into a cell a promoterless polynucleotide construct comprising a marker peptide-encoding sequence and a splice acceptor site upstream of the marker such that, upon integration of the construct into an actively transcribing region of the genome, the marker is expressed as a fusion protein with whatever protein is encoded by the gene. Expression is detected and/or quantified by, for example, fluorescence activated flow cytometry (FACS). Once expression is detected and/or quantified, the DNA sequence of the native gene into which the marker has been inserted, or the portion thereof, is determined. Once the sequence is obtained, it is compared to a sequence database for the identification of the protein, or portion thereof, encoded by such sequence (i.e., BLAST analysis).

According to the inventors of International Application No. PCT/US01/08770 (International Publication WO 01/70948), determination of the sequence of the native gene into which the marker has been inserted is accomplished by one of two different methods. In the first method, genomic DNA is recovered from the cell, and subjected to a restriction enzyme that cuts somewhere inside the marker, the sequence of which is known, and cuts somewhere upstream of the marker in the native gene. The resultant fragment, containing the 5′ portion of the marker and a portion of the native gene, is then self-ligated. The fragment is then amplified utilizing inverse PCR with primers generated from the known marker sequence, and the portion of the fragment containing the unknown gene, or a sub-portion thereof, is sequenced.

In the second method, mRNA is obtained from the cell, and reverse transcribed into complementary DNA (cDNA). The cDNA is then subjected to a restriction enzyme that recognizes a specific sequence that has been engineered into the construct between the start codon of the marker and the splice acceptor, but which actually cuts the unknown gene (exon) at a variable distance from the junction of the splice acceptor and the splice donor of the exon. A labeled primer generated from the known marker sequence is then utilized to extend the single-stranded DNA into the exon, followed by poly-dT tailing by terminal transferase. An oligo-dA primer is then used, in conjunction with a marker-specific primer, to amplify the 3′ portion of the exon. The amplified fragments are then ligated together end-to-end to create a concatamer, which is then sequenced.

While the above methods are useful for elucidating a protein profile for a given cell, such methods do not simultaneously provide any information regarding the quantity of the transcription level of a given gene since each length of the sequence tag generated by these methods is different, which is subjected to the PCR-bias that favors amplification of shorter sequence tags.

An alternative method for characterization of transcriptional profiling using the acquisition of short sequence tags from the 3′ end of mRNA transcripts has been described by others: “Serial Analysis of Gene Expression”, Velculescu et al, Science 1995 270:484; “Using the transcriptome to annotate the genome”, Saha et al., Nature Biotech 2002 19: 508; “Generation and analysis of melanoma SAGE libraries: SAGE advice on the melanoma transcriptome”, Weeraratna et al, Oncogene 2004 1:11. The SAGE method consists in digesting total double stranded cDNA with a 4-bp restriction enzyme that cuts at random positions within the cDNA and ligation of linkers to the restriction fragments located at the most 3′ end of the transcript, closest to the polyA sequence. These linkers contain a recognition sequence for a Type IIS restriction enzyme that will cut outside of its recognition sequence to generate a restriction fragment consisting in the linker sequence fused to 10-20 bp sequence of the 3′ end of the cellular mRNA. These tags are ligated together, and ditags are amplified by PCR, cloned and sequenced. Determination of the frequency of each tag is used to estimate the relative levels of gene expression for each transcript. The advantage of this method is that it allows for the simultaneous quantitative analysis of large number of transcripts without previous tagging. The disadvantage of this method is that the short sequence tags that are generated (12-14 bp in most cases) do not allow a precise assignment of the tag to a particular genomic locus, which precludes the identification of the gene that is being quantified. Recent improvements in this technology have pushed the length of the tag to 17 bp, followed by 4 bp of constant sequence corresponding to the recognition sequence of the first restriction enzyme which takes the total length of the tag to 21 bp. Analyses of human genome sequence have shown that 75% of 21 bp tags happen only once in the genome and can therefore be uniquely assigned to a single genomic locus. That means that even with the latest improvements, 25% of those tags still cannot be uniquely assigned to a single genomic locus. Moreover, as information is obtained only from the 3′ end of each transcript, it does not allow characterizing the alternative spliced forms of transcripts. This is important because alternative splicing is greatly responsible for gene expression complexity and protein diversity. In fact, some genetic diseases and cancers have been related to abnormal alternative splicing. Given the dearth of available information regarding exon-exon and exon-intron boundaries and the importance of such information, it would be desirable to obtain methods for elucidating a transcriptional profile of a given cell, wherein the methods simultaneously provide sequence information corresponding to the boundaries of exons of the genes encoding the proteins contained within the cell.

### BRIEF SUMMARY OF THE INVENTION

The present invention relates to a method for elucidating a transcriptional profile for a cell comprising providing a cell inserting at random positions into the cell's DNA a promoterless polynucleotide construct, wherein the construct comprises: a) a functional marker exon sequence flanked by functional 5′ splice acceptor and 3′ splicing donor consensus sequences, which define the 5′ and 3′ ends of the marker exon, respectively; b) a first Type IIS restriction enzyme recognition (RER) site, wherein the first Type IIS RER site (RER#1) is located at the 5′ end of the marker exon and oriented so it will cut a certain length upstream of its binding recognition sequence into the cellular exon fused to the marker exon that results after transcription and RNA splicing; c) a second Type IIS RER site (RER#2), wherein the second Type IIS RER site is located at the 3′ end of the marker exon and will cut at a certain length of base pairs downstream from its binding recognition site into the cellular exon sequence placed in that position after mRNA splicing; d) a third RER site (RER#3), which can be of Type II or Type IIS, located between the 5′ end of the marker gene and the RER#1 (FIG. 1B), or alternatively, downstream of RER#1 site (FIG. 1A); e) a fourth RER site, (RER#4) which can be Type II or Type IIS, located between the 3′ end of the marker gene and the second Type IIS RER site (RER#2) (FIG. 1B) or upstream of the second Type IIS RER site which is adjacent to 3′ end (FIG. 1A); f) a splice acceptor site located upstream of the first Type IIS restriction enzyme recognition site, and g) a splice donor site located downstream of the second Type IIS restriction enzyme recognition site, such that, upon integration of the construct into an actively transcribing region of the cell's genome, the marker exon is incorporated into the spliced mRNA of the tagged gene (step 1); isolating mRNA from the cell (step 2); reverse transcribing the isolated mRNA into cDNA (step 3); subjecting the cDNA to digestion with a Type IIS restriction enzyme (step 4) that recognizes each of the first and second Type IIS restriction enzyme recognition sites and thereupon cleaves the cDNA upstream of the first Type IIS restriction enzyme recognition site and downstream of the second Type IIS restriction enzyme recognition site such that a cDNA fragment is produced comprising the marker exon, and portions of the upstream and downstream cellular exon sequences (exon tags). The next steps of the method consist in self-ligating the cDNA fragment containing the marker exon (step 5) so that the flanking cellular exon tags are ligated together into an inverted di-tag configuration; followed by amplification by inverted PCR (step 6) of the di-tags with primers complementary to the marker exon sequence; subjecting the amplified di-tags to digestion with one or more restriction enzymes that recognize the third and fourth RER sites (step 7) such that the sequences corresponding to the marker exon is cleaved away from the di-tag fragment. These ditags can be directly cloned into sequencing vectors (step 8A), and sequenced individually (step 9A), or the whole population of ditags can be ligated together to form higher order polymers containing 2 or more di-tags (step 8B), and then cloned into sequencing vectors (step 9B). After obtaining the sequence of di-tags, the sequence data is compared against a genomic or cDNA sequence database such that the transcript tagged by the marker exon is identified (step 10). There are several steps in the data aggregation and analysis process. The first step consists in the classification of different di-tags into separate subgroups, and counting the frequency at which each di-tag shows up in the total population of di-tags (step 11). The second step consists in the comparison of the sequence of individual tags against a genomic or cDNA sequence database (step 12). As each half of the ditag corresponds to one exon fused to the marker exon by the process of splicing, the two halves of each ditag are supposed to be co-linear in the genomic DNA sequence or in the corresponding RNA. If they were not co-linear, they may represent an intermolecular ligation event that took place in step 5 of the method. The transcriptional level of a gene is therefore digitized and represented by the frequency of a given gene being sequenced. The alternative splicing information of a given gene can be obtained by comparing the exon pairs (upstream exon and downstream exon) acquired from each ditag of a given gene.

As the length of each tag fused to the marker exon can reach up to 20 bp, and two tags that are co-linear in the genome are obtained per di-tag, the method yields total length of 40 bp per ditag that can be used to determine the identity of the gene being quantified. This dual tag length allows unique genomic assignment to almost every mRNA being studied. Moreover, several ditags can be obtained per gene as retroviral integrations can happen at several locations within each gene. Therefore, expression of each gene can be quantified using several independently obtained di-tags, which gives more statistical significance and validity to the method. Another advantage of the method of the present invention over SAGE is that the present method does not rely on transcript polyadenylation for its identification.

If the marker exon encodes a protein that is in the proper translational frame with the upstream and downstream exons, the protein level of a given gene can also be quantified by the expression level of the resulting fusion protein.

Therefore, this invention is based on 6 principles:

1) An exon donor cassette based on gene trapping strategy to acquire bona fide exons.

2) A short sequence tag (14-20 bp) obtained from each exon trapped at both ends of the exon donor is ligated into a ditag for identification of a transcript and possible alternative splicing between exons.

3) Sequence ditags can be linked together to form long DNA molecules (concatamers) that can be cloned and sequenced. Sequencing of the concatamer clones results in the identification of individual tags.

4) The expression level of the transcript is quantified by the number of times a particular ditag is observed.

5) The bona fide exon boundaries can be used to annotate the human genome and for gene discovery. With the evidences of marker-cellular fusion protein, the existence of the translation of a hypothetic protein can be proved.

In one embodiment, the native sequence from the portion of the upstream sequence is of the same nucleotide length as the native sequence from the portion of the downstream sequence.

Preferably, digestion of the amplified di-tag fragments with the enzymes that recognize the third and fourth RER sites, and prior to sequencing, the multiple amplified di-tag fragments are ligated together to form a concatamer, wherein the concatamer is separated from others by cloning into a sequencing vector and then transforming into a single bacteria or by length fractionation and then is sequenced individually.

In another embodiment, the di-tag cDNA fragment is amplified by the primers with another Type-IIS recognition sequence at the 5′ end of the primer which can be used to digest away the primer from the ditag after PCR amplification. This will leave the smallest ditag fragment for latter concatamer ligation and sequencing.

In a further embodiment, the promoterless polynucleotide construct can be directly delivered into a cell with a transfection method or within a vector, which includes but is not limited to, a viral vector. Preferably, the viral vector is selected from the group consisting of a retroviral vector, a lentiviral vector, adeno-associated viral vector. Most preferably, the viral vector is a retroviral vector from the lentiviridiae family such as human immunodeficiency virus type 1 (HIV-1), as it has been shown that these viral vectors can integrate into actively transcribed genomic regions, with no particular preference for the position of integration within each transcriptional unit (“Transcription start regions in the human genome are favored targets for MLV integration”, We et al, Science 2003 300: 1749; “HIV-1 integration in the human genome favors active genes and local hotspots”

### BRIEF DESCRIPTION OF THE DRAWINGS

FIG. 1A is a schematic of a polynucleotide construct useful for the invention. In this example, integration of the marker gene can occur either in an intron or exon in split genes encoding protein products (including, but not limited to, e.g., genes without introns that encode proteins such as histones, etc., or genes encoding physiologically active RNAs, e.g., snRNA, scRNA, spliceosome components, etc.). For the sake of clarity, integration into an intron sequence of a cellular gene encoding a protein is shown. Placement of a splice acceptor (SA, i.e., human gamma-globin intron #2 splicing acceptor) upstream of the marker exon and a splice donor (SD, i.e., synthetic splice donor) downstream of the marker exon results in the synthesis of a mRNA encoding a fusion transcript that includes the marker exon fused to cellular sequences corresponding to upstream and downstream exons (occurs when the splice donor of the nearest upstream exon (closer to the start of transcription) is reacted with the splice donor slightly upstream of the marker, and when the splice donor slightly downstream of the marker is reacted with the splice acceptor of the nearest downstream exon. The construct further comprises a first Type IIS restriction enzyme recognition (RER#1) site (i.e., BsmFI or MmeI) located at the 5′ end of the marker immediately downstream of the SA, a second Type IIS RER site (RER#2) (i.e., BsmFI or MmeI) located at the 3′ end of the marker, immediately upstream of the SD, and two RER sites, RER#3 and RER#4 (i.e., NobI, BamHI), located immediately downstream of RER#1 and upstream of RER#2, respectively. FIG. 1B illustrates the alternative arrangement of theses RERs. In this case, RER#3 is between the SA and RER#1 and RER#4 is located between RER#2 and SD. In this case, the condition that has to be met is that RER#1 and RER#3 are sufficiently close to each other and to the 5′ end of the marker exon that the Type IIS enzyme that recognizes RER#1 is able to cut upstream to the 5′ end of the exon. Accordingly, the same condition has to be met on the 3′ end of the marker exon with RER#2 and RER#4.

FIG. 2 depicts a diagram of retroviral vectors based on MoMLV which enables the identification of exon boundaries in genes. pGT13 contains the gene encoding for Renilla reniformis green fluorescent protein (hrGFP) as the exon marker, defined by consensus splice acceptor and splice donor sequences. In this vector, the Type IIS RER sites RER#1 and RER#2 are recognized by the enzyme BsmFI, while RER#3 is recognized by NcoI and RER#4, by HindIII. pGTfs0-M contains the hrGFP gene preceded by a splice acceptor sequence and followed by the bovine growth hormone polyadenylation sequence. Incorporation of this vector into transcriptional units will render fusions of cellular exons upstream of the marker exon only and will introduce premature transcriptional termination. In this vector, RER#1 is recognized by MmeI, and RER#3, by NheI. LTR=long terminal repeat; NeoR=neomycin resistant gene;; BGHpA=bovine growth hormone poly-A signal; SA=splice acceptor (i.e. human gamma-globin intron #2 splicing acceptor); SD=splice donor (i.e. synthetic splice donor).

FIGS. 3A and 3B are schematics depicting the method of Serial Analysis of Vector Integration (SAVI) for elucidating a transcriptional profile for a given cell that permits the simultaneous identification of exon-intron boundaries. In this method, the construct that is inserted into the cell comprises a marker exon, two Type IIS restriction enzyme recognition (RER) sites located at both ends of the marker and two internal RER sites located close to the first Type IIS RER sites. The distribution of RER sites is as described in FIG. 1A. During splicing, assuming that the construct has integrated into an intron, the introns will be removed by the splicing mechanism in a given cell. Then, mRNA is isolated from the cell, and reverse transcribed into double stranded cDNA. The cDNA is subjected to a Type IIS restriction enzyme (RE) that recognizes RER#1 and RER#2 sites and thereupon cleaves the cDNA upstream of the first Type IIS RER site (RER#1) and downstream of the second Type IIS RER site (RER#2) such that a cDNA fragment is produced comprising the marker, and portions of the upstream and downstream exon flanking sequences (exon tags). Following digestion with the appropriate Type IIS RE, the fragment is self-ligated, and the self-ligated fragment is amplified by inverse PCR using marker-specific primers. Following amplification, the fragments are subjected to one or more restriction enzymes that recognize RER#3 and RER#4 sites and thereupon cleave the fragments such that the marker is cleaved away from the fragments. Following non-Type IIS RE digestion, the fragments are ligated together to form a concatamer, cloned into a bacterial sequencing vector and then sequenced by appropriate methods. The sequence is then compared to a sequence database such that the RNA transcript encoded by the sequence is identified. As can be appreciated by one of ordinary skill in the art, since each length of the ditag of upstream and downstream exon boundaries of each gene captured by this method is the same, PCR amplification still preserves the relative abundances of mRNA transcripts and the frequency of a ditag being amplified and sequenced. Therefore, the frequency of a ditag being sequenced can represent the level of transcription and mRNA abundance levels for a given gene. The combination of different exon boundaries in the ditags from the same gene will provide information about alternative splicing for that given gene.

FIGS. 4A and 4B illustrate the method of 5′SAVI, for elucidating a transcriptional profile for a given cell that permits the simultaneous identification of exon-intron boundaries. In this method, the construct that is inserted into the cell comprises a marker exon, two Type IIS restriction enzyme recognition (RER) sites located at both ends of the marker and two internal RER sites located close to the first TypeIIS RER sites. The distribution of RER sites is as described in FIG. 1A. During splicing, assuming that the construct has integrated into an intron, the introns will be removed by the splicing mechanism in a given cell. Then, mRNA is isolated from the cell, and reverse transcribed into double stranded cDNA. This method differs from the method illustrated in FIGS. 3A and 3B in that double stranded cDNA is synthesized only for RNA molecules bearing the marker sequence. The cDNA is subjected to a Type IIS restriction enzyme (RE) that recognizes the RER#1 site and thereupon cleaves the cDNA upstream of the first Type IIS RER site such that a cDNA fragment is produced comprising the marker, and portions of the upstream exon flanking sequence. Following digestion with the appropriate Type IIS RE, a linker is ligated and the exon tag is amplified by PCR using primers specific for the linker and for the marker. Following amplification, the fragments are subjected to one or more restriction enzymes that recognize RER#3 and an additional RER site present in Primer #2, such that the marker is cleaved away from the fragments. Next, the fragments are ligated together to form a concatamer, cloned into a bacterial sequencing vector and then sequenced by appropriate methods. The sequence is then compared to a sequence database such that the RNA transcript encoded by the sequence is identified.

FIGS. 5A and 5B illustrate the method of 3′SAVI, for elucidating a transcriptional profile for a given cell that permits the simultaneous identification of exon-intron boundaries. In this method, the construct that is inserted into the cell comprises a marker exon, two Type IIS restriction enzyme recognition (RER) sites located at both ends of the marker and two internal RER sites located close to the first TypeIIS RER sites. The distribution of RER sites is as described in FIG. 1A. During splicing, assuming that the construct has integrated into an intron, the introns will be removed by the splicing mechanism in a given cell. Then, mRNA is isolated from the cell, and reverse transcribed into double stranded cDNA. This method differs from the method illustrated in FIGS. 3A and 3B in that double stranded cDNA is synthesized only for RNA molecules bearing the marker sequence. The cDNA is subjected to a Type IIS restriction enzyme (RE) that recognizes the RER#2 site and thereupon cleaves the cDNA downstream of the second Type IIS RER site such that a cDNA fragment is produced comprising the marker, and portions of the downstream exon flanking sequence. Following digestion with the appropriate Type IIS RE, a linker is ligated and the exon tag is amplified by PCR using primers specific for the linker and for the marker. Following amplification, the fragments are subjected to one or more restriction enzymes that recognize RER#4 and an additional RER site present in Primer #2, such that the marker is cleaved away from the fragments. Next, the fragments are ligated together to form a concatamer, cloned into a bacterial sequencing vector and then sequenced by appropriate methods. The sequence is then compared to a sequence database such that the RNA transcript encoded by the sequence is identified.

### DEFINITIONS

Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described below are those well known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see, generally, Molecular Cloning: A Laboratory Manual, 3^{rd} ed., Sambrook et al., Cold Spring Harbor Laboratory Press, Cold Spring Harbor, N.Y. (2001), which is incorporated herein by reference) which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. As employed throughout the disclosure, the following terms, unless otherwise indicated, shall be understood to have the following meanings and are more fully defined by reference to the specification as a whole:

As used herein, the term “cell” is intended to refer to any eukaryotic or prokaryotic cell containing genetic material, including, but not limited to, those of microorganisms, plants, invertebrates, vertebrates, and mammals.

As used herein, the term “inserting” is intended to refer to the incorporation of a composition, such as a polynucleotide, into the genome of a eukaryotic or prokaryotic cell. The term is also intended to encompass terms such as “transformation,” “transfection,” and “transduction” as those terms are understood in the art.

As used herein, the term “promoter” is intended to refer to a region of DNA upstream of the transcription start site of a given gene, and which is involved in recognition and binding of RNA polymerase and other proteins to initiate transcription.

As used herein, “polynucleotide” is intended to refer to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. The term further encompasses DNAs or RNAs with backbones modified for stability or for other reasons. Moreover, DNAs or RNAs comprising unusual bases, such as inosine, or modified bases, such as tritylated bases, to name just two examples, are encompassed by the term “polynucleotides.” It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

As used herein, the term “promoterless polynucleotide construct” is intended to refer to a polynucleotide that does not comprise a promoter sequence such that the marker gene included within the construct cannot be expressed unless the construct becomes integrated into an actively transcribing region of a cell's genome.

As used herein, the term “marker exon” or “marker” is intended to refer to a polynucleotide sequence that may or may not encode a protein. For the purpose of this invention, there is provided a functional exon with enough space to accommodate primers for inverse PCR, Type IIS and non-Type IIS RERs. This marker exon can encode for a protein marker such as a fluorescent protein, lacZ, which encodes β (beta) -galactosidase, gus, which encodes β-glucuronidase, and luc, which encodes luciferase or an epitope that can be recognized by an antibody or other detection reagent to detect for molecular modification including, but not limited to, protein glycosylation, kinase, phosphatase reactions etc. In the methods of the invention, marker gene expression can be detected by any suitable means known in the art or developed in the future. Ultimately, however, detection of marker gene expression will depend on the chemical and/or physical characteristics of the fusion protein encoded resulting after integration of the marker exon within a cellular transcriptional unit. Preferably, and as indicated herein, the marker gene encodes a protein capable of fluorescing, and detection of the protein is preferably accomplished by fluorescence activated flow cytometry. In addition to detection of the presence of a protein in a cell, it may be desirable to quantify the protein. Quantification of the protein can also be accomplished by fluorescence activated flow cytometry.

As used herein, the term “exon tag” or “tag” refers to a short polynucleotide sequence fused to the exon marker gene that serves as a sequence identifier of the RNA transcriptional unit that was “marked” or “tagged” by insertion of the marker exon. In eukaryotic cells the exon tags correspond to the exon-intron junctions of cellular exons, and identify the terminal sequence of a cellular exon, that is fused to the marker exon by the process of RNA splicing. In prokaryotic cells the tag identifies the RNA transcript where the marker exon was inserted.

As used herein, the terms “encodes,” “encoding” or “encoded,” with respect to a specified nucleic acid, are meant to comprise the information for translation into a specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

As used herein, the term “restriction enzyme” is intended to refer to a nuclease that is able to recognize and cut specific sequences in DNA. A sequence that is recognized by a particular restriction enzyme is a “restriction enzyme recognition site.” As used herein, a “Type IIS restriction enzyme recognition site” is a DNA sequence that is recognized by a Type IIS restriction enzyme. Type IIS restriction enzymes include, for example, BsmFI, FokI, MmeI, BsgI, and AlwI. Type IIS restriction enzymes generally cleave outside of their recognition sequence to one side. These enzymes are intermediate in size, and recognize sequences that are continuous and asymmetric. They comprise two distinct domains, one for DNA binding, and the other for DNA cleavage. Type IIS restriction enzymes are thought to bind to DNA as monomers for the most part, but to cleave DNA cooperatively, through dimerization of the cleavage domains of adjacent enzyme molecules. As used herein, a “non-Type IIS restriction enzyme recognition site” is a sequence that is not recognized by a Type IIS restriction enzyme, but that is recognized by another restriction enzyme, such as a Type II restriction enzyme. Type II restriction enzymes include, for example, BamHI, HindIII, NcoI, NotI, etc. Type II restriction enzymes cut DNA at defined positions close to or within their recognition sequences. The most common type II enzymes are those that cleave DNA within their recognition sequences. Enzymes of this kind are the most common ones available commercially. Most recognize DNA sequences that are symmetric because they bind to DNA as homodimers, but a few recognize asymmetric DNA sequences because they bind as heterodimers. Some enzymes, such as EcoRi, recognize continuous sequences (GAATTC) in which the two half-sites of the recognition sequence are adjacent, while others, such as BglI, recognize discontinuous sequences (GCCNNNNNGGC)(SEQ ID NO:1) in which the half-sites are separated. Cleavage leaves a 3′-hydroxyl on one side of each cut and a 5′-phosphate on the other. Type II restriction enzymes tend to be small in size, with subunits in the 200-350 amino acid range.

As used herein, the term “splice acceptor site” is intended to refer to any individual functional splice acceptor or functional splice acceptor consensus sequence that permits the construct of the invention to be processed such that it is included in any mature, biologically active mRNA, provided that it is integrated in an active chromosomal locus and transcribed as a contiguous part of the pre-messenger RNA of the chromosomal locus. An example of splice acceptor consensus sequences for mammalian cells is (Y)₁₋₁₀NCAG.

As used herein, the term “splice donor site” is intended to refer to any individual functional splice donor or functional splice donor consensus sequence that permits the construct of the invention to be processed such that it is included in any mature, biologically active mRNA, provided that it is integrated in an active chromosomal locus and transcribed as a contiguous part of the pre-messenger RNA of the chromosomal locus. An example of splice donor consensus sequences for mammalian cells is GTRAGT.

As used herein, the term “isolating,” in reference to nucleic acid material, is intended to refer to the extraction from a cell of nucleic acid material such that the material is substantially free from components that normally accompany or interact with it as found in its naturally occurring environment. Methods for isolating nucleic acid material from cells are well-known in the art. See, generally, Molecular Cloning: A Laboratory Manual, 3^{rd} ed., supra.

As used herein, the phrase “reverse transcribing,” in reference to mRNA that has been isolated from a cell, is intended to refer to the conversion of cellular mRNA to DNA. Following such a conversion, the DNA is referred to as complementary DNA, or cDNA. Methods for reverse transcribing mRNA into cDNA are well-known in the art. See, generally, Molecular Cloning: A Laboratory Manual, 3^{rd} ed., supra.

As used herein, the term “upstream,” of any particular point reference (i.e., marker gene, exon, transcription start site, splice acceptor, translational start codon) refers to the region occurring 5′ of that reference point. If no point of reference is given, “upstream” is meant to be interpreted taking as reference the 5′ to 3′ direction of transcription of the gene or RNA in question.

As used herein, the term “downstream,” of any particular reference point (i.e., marker gene, exon, transcription start site, splice donor, translational stop codon) is intended to refer to the region occurring 3′ to that particular reference point. If no point of reference is given, “downstream” is meant to be interpreted taking as reference the 5′ to 3′ direction of transcription of the gene or RNA in question.

As used herein, the term “native sequence” or “cellular sequence” refers to the naturally-occurring genomic sequence of a particular cell.

As used herein, the term “ligating,” with reference to a linear nucleic acid molecule(s), such as DNA, is intended to refer to the creation of a phosphodiester bond between one end of a first linear nucleic molecule and one end of a second linear nucleic acid molecule, such that a single, linear nucleic acid molecule is produced. As used herein, the term “self-ligating” is intended to refer to the creation of a phosphodiester bond between one end of a linear nucleic acid molecule and the other end of the same molecule. Methods for ligating and self-ligating nucleic acid molecules are well-known in the art. See, generally, Molecular Cloning: A Laboratory Manual, 3^{rd} ed., supra.

As used herein, the term “sequencing,” with reference to a nucleic acid molecule, such as DNA, is intended to refer to the elucidation of the composition and order of the nucleotides making up the nucleic acid molecule. Methods of sequencing are well-known in the art, and include, for example, PCR chain termination, the methods of Sanger, or those of Maxam and Gilbert. See, generally, Molecular Cloning: A Laboratory Manual, 3^{rd} ed., supra.

As used herein, the term “amplified” is intended to refer to the construction of multiple copies of a nucleic acid sequence or multiple copies complementary to the nucleic acid sequence using at least one of the nucleic acid sequences as a template. Amplification systems include the polymerase chain reaction (PCR) system (see, e.g., U.S. Pat. No. 4,683,195, the disclosure of which is incorporated herein by reference), ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., eds., American Society for Microbiology, Washington, D.C. (1993).

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide,” “peptide,” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of post translation events, including natural processing event and events brought about by human manipulation which do not occur naturally. Circular, branched and branched circular polypeptides may be synthesized by non-translation natural process and by entirely synthetic methods, as well.

As used herein, the term “operably linked” includes reference to a functional linkage between a promoter and a second sequence, wherein the promoter sequence initiates and mediates transcription of the DNA sequence corresponding to the second sequence. Generally, “operably linked” means that the nucleic acid sequences being linked are contiguous and, where necessary to join two protein coding regions, contiguous and in the same reading frame.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et a., Nucleic Acids Res. 25:3389-3402 (1997). Software for performing BLAST analyses is publicly available, e.g., through the National Center for Biotechnology-Information-n (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Appl. Biol. Sci. 4:11-17 (1988), e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, ore preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

(e)(ii) The terms “substantial identity” in the context of a peptide indicates that a peptide comprises a sequence with at least 70% sequence identity to a reference sequence, preferably 80%, ore preferably 85%, most preferably at least 90% or 95% sequence identity to the reference sequence over a specified comparison window. Optionally, optimal alignment is conducted using the homology alignment algorithm of Needleman and Wunsch, J. Mol. Biol. 48:443 (1970). an indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

### DETAILED DESCRIPTION OF THE INVENTION

The methods by which the objects, features and advantages of the present invention are achieved will now be described in more detail. These particulars provide a more precise description of the invention for the purpose of enabling one of ordinary skill in the art to practice the invention, but without limiting the invention to the specific embodiments described.

The present invention relates to a method, denominated Serial Analysis of Vector Integration (SAVI), for elucidating a transcriptional profile for a cell by inserting at random positions into the cell's genome a promoterless polynucleotide construct so that the marker exon or marker sequence becomes part of a functional transcriptional unit. The polynucleotide sequence can be integrated at random positions into the target cell's genome by any means known in the art such as DNA transfection, transduction mediated by retroviral vectors, in vivo recombination, DNA transposition or retrotansposition.

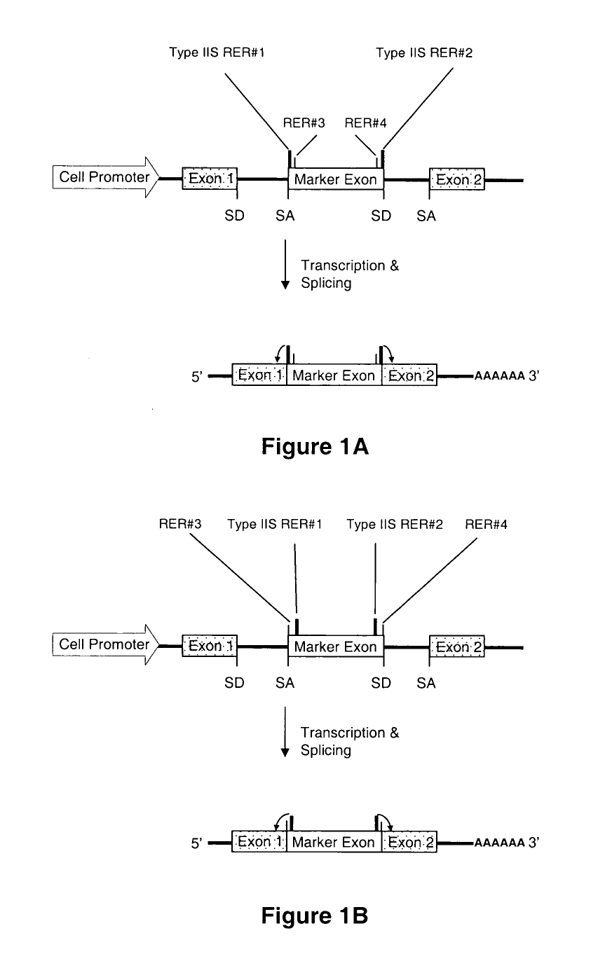

In a preferred embodiment, the polynucleotide can be inserted into eukaryotic cells that are proficient at mRNA splicing. In this most preferred embodiment, the marker sequence would be defined by flanking splice acceptor and splice donor consensus sequences, so that after RNA splicing, the marker sequence (marker exon) would be incorporated as an additional exon in the mature mRNA. In the case where integration of the marker sequence in the final mature RNA is dependent on RNA splicing the preferred structure of the marker exon sequence would be the one shown in FIG. 1A or FIG. 1B. Briefly, this structure consists in a 5′ to 3′ orientation in (FIG. 1A): a) a functional 5′ splice acceptor consensus sequence, b) a Type IIS RER site, oriented so it can cleave the DNA fused upstream of the 5′ end of the marker exon (RER#1), c) a Type IIS or non-Type IIS RER site (RER#3), d) a polynucleotide sequence corresponding to the marker exon, e) a Type IIS or non-Type IIS RER site (RER#4), f) a Type IIS RER site oriented so that it can cleave sequences located downstream of the 3′ end of the marker exon (RER#2), g) a splice donor consensus sequence. Alternatively, the structure can consist in a 5′ to 3′ orientation in (FIG. 1B): a) a functional 5′ splice acceptor consensus sequence, b) a Type IIS or non-Type IIS RER site (RER#3), c) a Type IIS or RER site, oriented so it can cleave the DNA fused upstream of the 5′ end of the marker exon (RER#1), d) a polynucleotide sequence corresponding to the marker exon, e) a Type IIS RER site oriented so that it can cleave sequences located downstream of the 3′ end of the marker exon (RER#2), f) a Type IIS or non-Type IIS RER site (RER#4), and g) a splice donor consensus sequence. In summary, at least one of the two RER sites located at each end of the marker exon has to be recognized by a Type IIS restriction enzyme. These RER sites have to be oriented in such a way that the Type IIS restriction enzyme cuts the DNA located outside the boundaries that define the marker exon, and located sufficiently close from the border of the marker exon so that after cutting into the flanking exons generates tags of 8 or more base pairs.

This invention, however, is not limited to eukaryotic cells that are proficient at RNA splicing but can also be applied to characterize the transcriptional profile of cells that do not depend on RNA splicing mechanisms, such as prokaryotic organisms, or to transcriptional units in eukaryotic cells that do not suffer RNA splicing such as histones RNA, or that have very small number and size of introns such as transcriptional units in fungi and other lower eukaryotes. In this embodiment, the marker sequence would not be defined by flanking splice acceptor and splice donor consensus sequences but it would consist in a linear DNA molecule flanked by two Type IIS restriction sites oriented so that the cutting sequences are located outside the boundaries of the marker sequence. In this case, the preferred structure of the polynucleotide marker gene would contain the elements defined by points b) to f) described above, and could actually integrate into a transcriptional unit in any orientation to produce equivalent results.

Any of the well known procedures for introducing the marker gene into host cells can be used to introduce a vector into cells. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra). For the generation of a transgenic cell, it is only necessary that the particular genetic engineering procedure used be capable of successfully introducing at least one transgene into at least one host cell, which can then be selected using standard methods. Methods of culturing prokaryotic or eukaryotic cells are well known and are taught, e.g., in Ausubel et al., Sambrook et al., 1993, and in Freshney, Culture of Animal Cells, 3. sup.rd. Ed., A Wiley-Liss Publication.

After the expression vector is introduced into the cells, the transfected cells are cultured under conditions favoring expression of the marker gene wherein the mRNA is recovered from the culture using standard techniques identified below. Methods of culturing prokaryotic or eukaryotic cells are well known and are taught, e.g., in Ausubel et al., Sambrook et al., and in Freshney, 1993, Culture of Animal Cells, 3. sup.rd. Ed., A Wiley-Liss Publication.

In prokaryotic cells, random integration of the marker gene into the target cell's genome can be mediated by DNA transfection of linear DNA, or by retrotransposons, transposons or phages that have been modified to include the flanking Type IIS RER sites at the ends of their linear molecules.

In eukaryotic cells, random integration of the marker gene into the target's cell genome can be mediated by DNA transfection of linear DNA, or by integrative viral vectors being retroviral vectors or adeno-associated vectors the most preferred choices. In a preferred embodiment the polynucleotide construct is included within an appropriate gene transfer vehicle which is then used to transduce cells to express the marker gene by the recipient host cells.

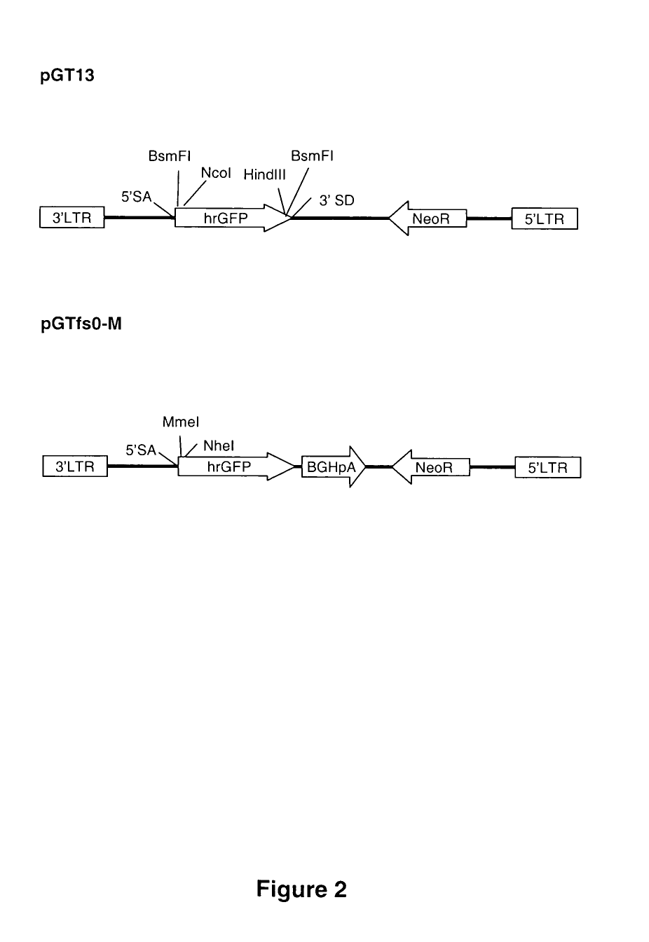

FIG. 2 shows examples of retroviral vector structures that have been used to practice the method of the invention in human cancer cells. In a preferred embodiment, the vector is a viral vector. Examples of suitable viral vectors include retroviral vectors (i.e., oncoretrovirus, lentivirus, foamy virus), and parvoviral vectors (i.e., adeno-associated virus). Preferably, the viral vector is a retroviral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, lentivirus, and mammary tumor virus.

Retroviral vectors have several properties that make them useful for gene transfer. First is the ability to construct a “defective” virus particle that contains the gene of interest and is capable of infecting cells but lacks viral genes and expresses no viral gene products. The MoMLV genome encodes the polyproteins gag, pol, and env that together constitute a retroviral particle. The gag and pol genes encode the inner core of the retrovirus as well as the enzymes required for processing the retroviral gene after infection of the target cell. The env gene forms the outer envelope of the virus and recognizes a specific receptor on target cells. To construct a retroviral vector the sequences encoding the viral proteins (Gag, Pol and Env) are integrated into a packaging cell line, and separated from the sequences necessary for transcription, packaging, reverse transcription and integration (5′LTRs, psi, PPT, 3′LTR). Retroviral vectors are capable of permanently integrating the genes they carry into the chromosomes of the target cell at random positions. Murine retroviral vectors are generally produced at titers of 10⁵-10⁶ cfu/ml and can accommodate an insert of about 7.5 kb of heterologous sequence. The marker exon can be incorporated into the proviral backbone in several general ways. The most straightforward constructions are ones in which the structural genes of the retrovirus are replaced by a single gene which then is transcribed under the control of the viral regulatory sequences within the long terminal repeat (LTR). In one embodiment, the retroviral vector may be one of a series of vectors described in Bender, et al., J. Virol. 61:1639-1649 (1987), based on the N2 vector (Armentano, et al., J. Virol., 61:1647-1650) containing a series of deletions and substitutions to reduce to an absolute minimum the homology between the vector and packaging systems. These changes have also reduced the likelihood that viral genes would be expressed. In the first of these vectors, LNL-XHC, the natural ATG start codon of gag was altered by site-directed mutagenesis to TAG, thereby eliminating unintended protein synthesis from that point. The vector LNL6 was made, which incorporated both the altered ATG of LNL-XHC and the 5′ portion of MoMuSV which obviates the expression of the amino terminal of pPr80gag. The 5′ structure of the LN vector series thus eliminates the possibility of expression of retroviral reading frames, with the subsequent production of viral antigens in genetically transduced target cells. In a final alteration to reduce overlap with packaging-defective helper virus, Miller has eliminated extra env sequences immediately preceding the 3′ LTR in the LN vector (Miller, et al., Biotechniques, 7:980-990, 1989). Miller, et al. have developed the combination of the pPAM3 plasmid (the packaging-defective helper genome) for expression of retroviral structural proteins together with the LN vector series to make a vector packaging system where the generation of recombinant wild-type retrovirus is reduced to a minimum through the elimination of nearly all sites of recombination between the vector genome and the packaging-defective helper genome (i.e., LN with pPAM3). In one embodiment, the retroviral vector may be a MoMLV of the LN series of vectors, such as those hereinabove mentioned, and described further in Bender, et al. (1987) and Miller, et al. (1989). Such vectors have a portion of the packaging signal derived from a mouse sarcoma virus, and a mutated gag initiation codon. The term “mutated” as used herein means that the gag initiation codon has been deleted or altered such that the gag protein or fragment or truncations thereof, are not expressed. Efforts have been directed at minimizing the viral component of the viral backbone, largely in an effort to reduce the chance for recombination between the vector and the packaging-defective helper virus within packaging cells. A packaging-defective helper virus is necessary to provide the structural genes of a retrovirus, which have been deleted from the vector itself. Helper viruses include, but are not limited to, retroviral AMIZ helper virus, or other retro elements (see, e.g., Young et al., J. Virol. 74(11):5242-9 (2000)), which can prevent the unwanted silencing of helper virus by cellular DNA methylation (see, e.g., Young et al., J. Virol. 74(7)3177-87 (2000)). The AMIZ helper virus-packaging cell line can produce vector titer up to 2×10⁷ CFU (colony formation units)/ml. In circumstances where the production of retrovirus is limited, alternative methods of retroviral production can be performed using a chimeric adenovirus system to produce vector titers up to 5×10⁹ cfu/ml (Ramsey et al., Biochem. Biophys. Res. Comm. 246(3):912-9 (1998); Caplen et al., Gene Ther. 6(3):454-9 (1999)). In a preferred embodiment, a retroviral vector packaging cell line is transduced with a retroviral vector containing the exon marker sequences. Examples of packaging cells which may be transfected include, but are not limited to the PE501, PA317, Ψ2, Ψ-PAM, PA12, T19-14X, VT-19-17-H2, ΨCRE, ΨCRIP, GP+E-86, GP+envAM 12, DAN and AMIZ cell lines. Methods for transfecting the retroviral vector DNA into retroviral packaging cell lines include, but are not limited to, electroporation, the use of liposomes, and calcium phosphate co-precipitation.

In another embodiment, the retroviral vectors can be based on human immunodeficiency virus Type I, using backbones for vector and helper packaging plasmids as described by Naldini et al, Science 1996, 272: 263-267; Zufferey et al., Nature Biotechnology 1997, 15: 871-875; and Reiser et al., Proc. Natl. Acad. Sci. USA 1996, 93: 15266-15271. Moreover, these vectors can withstand a deletion in the 3′ U3 region of the 3′ LTR that turns them into self-inactivating vectors after integration into the target genome, without a negative impact in vector titers (Zufferey et al., J of Virology 1998, 72: 9873-9880; Miyoshi et al., J. of Virology 1998, 72: 8150-8157). Lentiviral vectors pseudotyped with the VSV-G envelope have the additional advantage of wide tropism and high efficiency of infection of dividing and non-dividing cells. Also, they can be produced at high titers (5×10⁶-10⁷ tu/ml) and have a larger cloning capacity than murine retroviral vectors.

It is also possible to insert into the cell the promoterless polynucleotide construct comprising the marker via a naked DNA delivery vector. Naked DNA delivery of a gene of interest is facilitated by receptor-mediated transfection or homologous recombination. In a homologous recombination embodiment, the naked DNA vector is engineered to have highly repeated sequences such as Alu flanking the marker gene so that recombination is facilitated at the repetitive sites causing integration of the nucleotide. Alu sequences are approximately 300 bp in length and are found on average every 3000 bp in the human genome. Delivery of naked DNA can be accomplished by standard methods including, but not limited to, lipid-mediated transfection (cationic, anionic, and neutral charged), activated dendrimers (PolyFect® Reagent, SuperFect® Reagent, Qiagen), polyethyleneimine (PEI)-mediated transfection, receptor-mediated transfection (fusogenic peptide/protein), calcium phosphate transfection, electroporation, particle bombardment, direct injection of naked DNA, diethylaminoethyl (DEAE-dextran transfection), etc. Though the preferred embodiment is the use of viral based vectors, the use of other high efficiency plasmid-based vectors is not precluded.

In a preferred embodiment, the vector comprises a selectable marker to enable the selection of transformants. The selectable marker can be, for example, an antibiotic resistance gene, such as those that confer resistance to G418, puromycin, hygromicin B and the like. These can include genes from prokaryotic or eukaryotic cells such as dihydrofolate reductase or multi-drug resistance I gene, hygromycin B resistance that provide for positive selection. Any type of positive selector marker can be used such as neomycin or Zeocyn and these types of selectors are generally known in the art. Several procedures for insertion and deletion of genes are known to those of skill in the art and are disclosed. See, e.g., Molecular Cloning: A Laboratory Manual, 3^{rd} ed., supra. An entire transcription unit must be provided for the selectable marker genes (promoter-gene-polyA) and the genes must be flanked on one end or the other with promoter regulatory region and on the other with transcription termination signal (polyadenylation site). Any known promoter/transcription termination combination can be used with the selectable marker genes. For example, promoters such as cytomegalovirus promoter or Rous Sarcoma Virus can be used in combination with various ribosome elements such as SV40 poly A. The promoter can be any promoter known in the art including constitutive, (supra) inducible, (tetracycline-controlled transactivator (tTA)-responsive promoter (tet system, Paulus et al., J. Virol. 70(1):62-7 (1996)), or tissue specific, (such as those cited in Costa et al., Euro. J. Biochem. 258:123-31(1998); Fleischmann etal., FEBS Letters 440:370-76 (1998); Fassati et al., Human Gene Ther. 9:2459-68 (1998); Valerie et al., Human Gene Ther. 9:2653-59 (1998); Takehito et al., Human Gene Ther. 9:2691-98 (1998); Lidberg et al., J. Biol. Chem. 273(47):31417-26 (1998); Yu et al., J. Biol. Chem. 273(49):32901-9 (1998)). These types of sequences are well known in the art and are commercially available through several sources, ATCC, Pharmacia, Invitrogen, Stratagene, Promega.

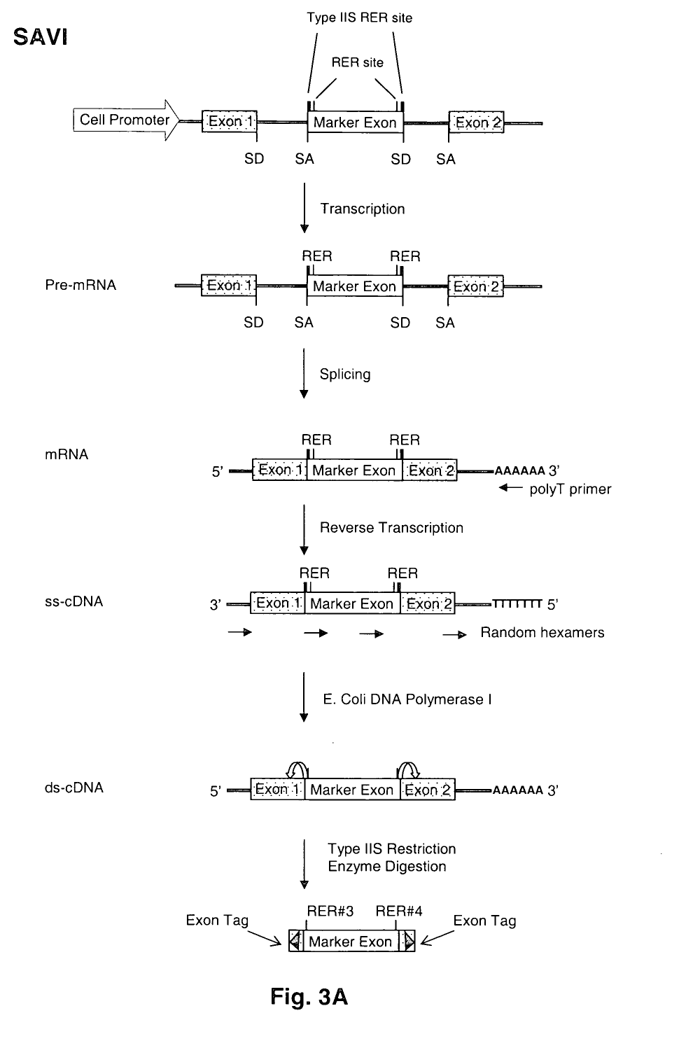

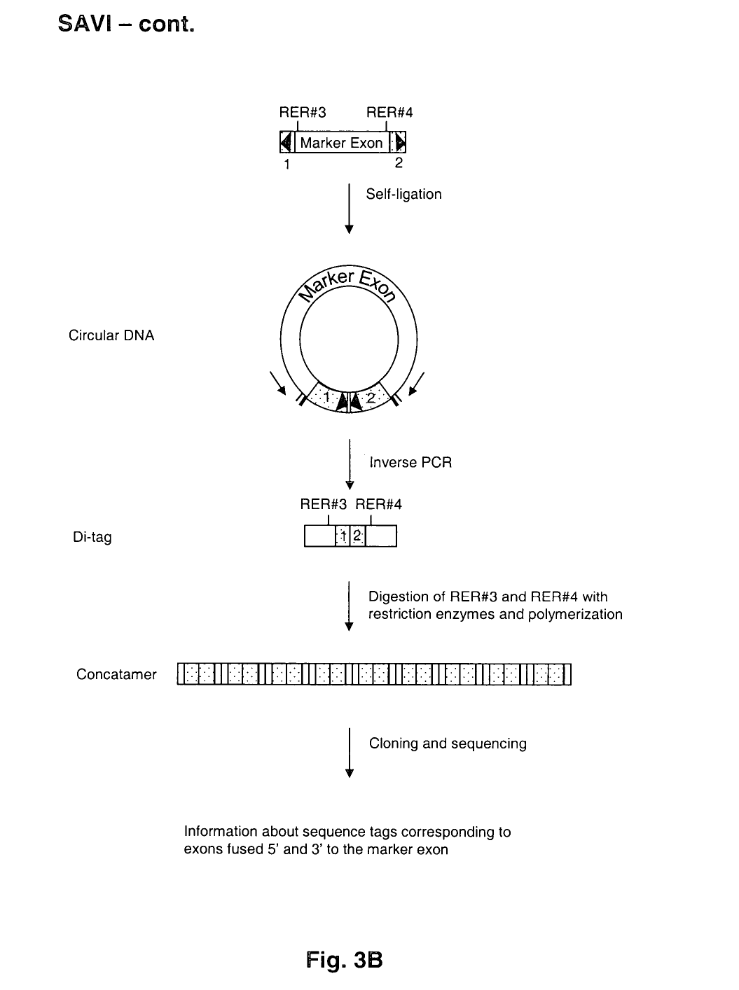

After the marker exon has been randomly integrated into the target cell's genome by any of the methods mentioned above, the next step involves the isolation of mRNA from the cell and reverses transcription to obtain a population of double stranded cDNA molecules (FIGS. 3A and 3B). Double stranded cDNA can be obtained from the whole population of purified RNA molecules or selectively from those molecules that have incorporated the marker exon sequences. Methods for purification of RNA from prokaryotic and eukaryotic cells and for synthesis of double stranded cDNA are well known and described in the art. See, generally, Molecular Cloning: A Laboratory Manual, 3^{rd} ed., supra.

Once the double stranded cDNA has been obtained, the next step of the method involves subjecting the cDNA to digestion with a Type IIS restriction enzyme that recognizes each of the first and second Type IIS RER sites located at each end of the marker exon and thereupon cleaves the cDNA upstream of the first Type IIS RER site and downstream of the second Type IIS RER site such that a cDNA fragment is produced comprising the marker exon, and portions of the upstream and downstream cellular exon sequences (exon tags). This results in a “di-tag” fragment comprising the marker, as well as captured sequences of DNA (tags) of predetermined size flanking each side of the marker. In a preferred embodiment, the “tags” are of equal size, i.e., 8, 10, 14, 20 nucleotides in length.

The next step of the method consists in self-ligating the cDNA fragment containing the marker exon so that the flanking cellular exon tags are ligated together in an inverted di-tag configuration.

The next step involves an amplification of the di-tags by inverted PCR (see, for example, Ochman et al., Genetics 120:621-625 (1988) and Triglia et al. (1988) Nucl. Acids Res. 16: 8186) with primers that anchor on the marker exon sequence. The conditions of the PCR and primers to be used depend on the particular sequence of the marker exon. As the length of all di-tags of the population is the same, the PCR amplification step does not introduce any bias towards any particular di-tag sequence, keeping constant the relative ratios and abundances of each di-tag within the total population. This permits using the frequencies of each sequenced tag as indicators of relative mRNA expression levels.

The next step involves purification of the amplified products away from the rest of the fragments. This is a straightforward step that can be performed by agarose or polyacrylamide gel electrophoresis and purification of the DNA band corresponding to the population of amplified tags. As mentioned above, all tags will have the same length and therefore will form a discrete band in the gel that can be distinguished from other cDNA fragments and non-specific PCR amplification products present in the mix. The size of this PCR band will be approximately 70-120 bp depending on the length of primers and the distance between their 3′ ends and the splice junction sites.

After the amplified population of di-tags has been amplified, it is subjected to digestion with one or more restriction enzymes that recognize the RER#3 and RER#4 sites and thereupon cleave the fragment such that the sequences corresponding to the marker exon is cleaved away from the fragment. If the primers used to amplify the di-tags are biotinylated, then the end fragments corresponding to the PCR amplification primers can be removed from the mix with magnetic beads coupled to streptavidin. Alternatively, the core fragment containing the di-tag and flanked by two short validation sequences (8-12 bp) corresponding to half of the recognition site of the Type II enzyme (3-4 bp) and the recognition site of the Type IIS enzyme (5-6 bp), can be purified away from the primer sequences by gel electrophoresis. Depending on the Type IIS restriction enzyme that was used and the particular design of the polynucleotide exon marker, this core fragment will have an approximate size of 40 bp to 80 bp.

These ditags can be directly cloned into sequencing vectors and sequenced individually or the whole population of ditags can be subjected to an additional step of ligation to form higher order polymers containing two or more di-tags per linear DNA molecule. This offers an advantage since theoretically, 15 di-tags of 50 bp each can be sequenced in a single sequencing reaction, which significantly accelerates the throughput.

The individual or polymerized di-tags can be cloned in any of numerous commercially available sequencing plasmid vectors such as pUC 18, pUC 19 (Stratagene), pBluescript (Stratagene), pLITMUS (New England Biolabs), pCR4-TOPO (Invitrogen), etc. The procedures for this step are well know for anyone skilled in the art, or can be followed according to the instructions provided by the plasmid supplier. See, generally, Molecular Cloning: A Laboratory Manual, 3^{rd} ed., supra.

After the di-tags are cloned, plasmid DNA can be purified and sequenced following well known protocols. See, generally, Molecular Cloning: A Laboratory Manual, 3^{rd} ed., supra. Alternatively, the DNA fragment containing the polymerized ditags can be directly amplified by PCR from bacterial colonies, with primers that anchor at both flanks of the multiple cloning site of the sequencing plasmid, and directly sequenced by the Sanger reaction. See, generally, Molecular Cloning: A Laboratory Manual, 3^{rd} ed., supra.

After obtaining the sequence of di-tags, the sequence data is compared against a genomic or cDNA sequence database such that the RNA transcript tagged by the marker exon is identified. There are several steps in the data aggregation and analysis process. The first step consists in the classification of different di-tags into separate subgroups according to their sequence (indexing), and determination of the frequency at which each tag or di-tag shows up in the total population of di-tags. The second step consists in the comparison of the sequence of each portion of the di-tag against a genomic or cDNA sequence database.

The database can consist of annotated or unannotated genomic sequences that find expression in cells as RNA (independent of their translation into protein, e.g., snRNA, scRNAs, RNAs with catalytic activities, etc.), cDNA libraries, EST libraries, protein sequence libraries (including DNA sequences (with or without intronic or exonic sequences) and amino-acid sequences (including primary, secondary and/or tertiary structure information)). Examples of such databases would include the publicly available EST and genomic databases. The end result of the matching step is that every tag becomes associated with a genetic unit (including subdivisions thereof such as specific intron or exon within a transcription unit) or becomes marked as an unknown so that it can be run again as more information about the proteome/transcriptome becomes known.

Comparison of each sequence tag to a nucleotide sequence database can be performed by any of several means known to operators skilled in the art, such as BLAST analysis. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-44 (1988); Higgins and Sharp, CABIOS 5:151-3 (1989); Corpet et al., Nucleic Acids Res. 16:10881-90 (1988); Huang et al., Computer Appl. Biosci. 8:155-65 (1992), and Pearson et al., Methods Mol. Biol. 24:307-31 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See Current Protocols in Molecular Biology, Chapter 19, Ausubel et al., eds., Greene Publishing and Wiley-Interscience, New York (1995). Software for performing BLAST analyses is publicly available, e.g., through the National Center for Biotechnology-Information (http://www.ncbi.nlm.nih.gov/).

As each half of the ditag corresponds to one exon fused to the marker exon by the process of splicing, the two halves of each ditag are supposed to be co-linear in the genomic DNA sequence or in the corresponding RNA. If they were not co-linear, they may represent an intermolecular ligation event that took place during the self ligation that takes place after digestion with the Type IIS restriction enzymes, and those di-tags are discarded from further comparison or re-run as independent tags. The transcriptional level of a gene is therefore digitized and represented by the frequency of tags sequenced that correspond to a given gene. Alternative splicing information of a given gene can be obtained by comparing the exon pairs (upstream exon and downstream exon) acquired from each di-tag of a given gene. The final output of the method is a database containing information about sequenced tags, the frequency of each tag within the total population, the gene from where that tag comes from, and alternative splicing information data.

In alternative embodiments of this method, instead of capturing both tags fused upstream and downstream of the marker exon, sequence tags fused to either side of the marker exon can be captured independently. In this case, isolated tags would contribute to information related to the relative abundance of each transcript, and also would identify intron-exon borders but would not provide information about alternative splicing.

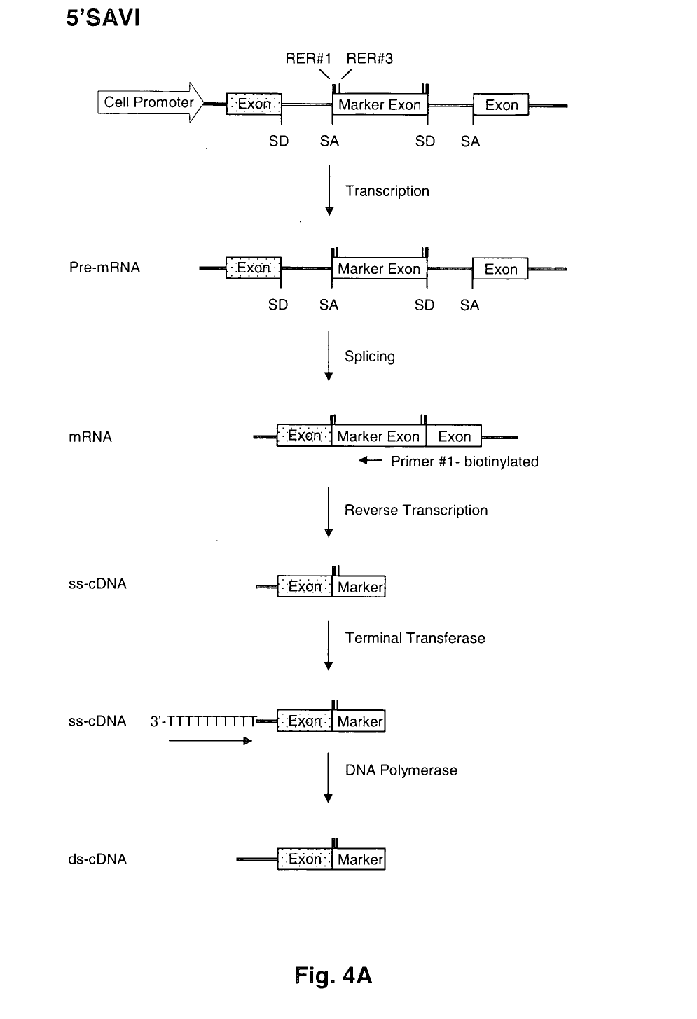

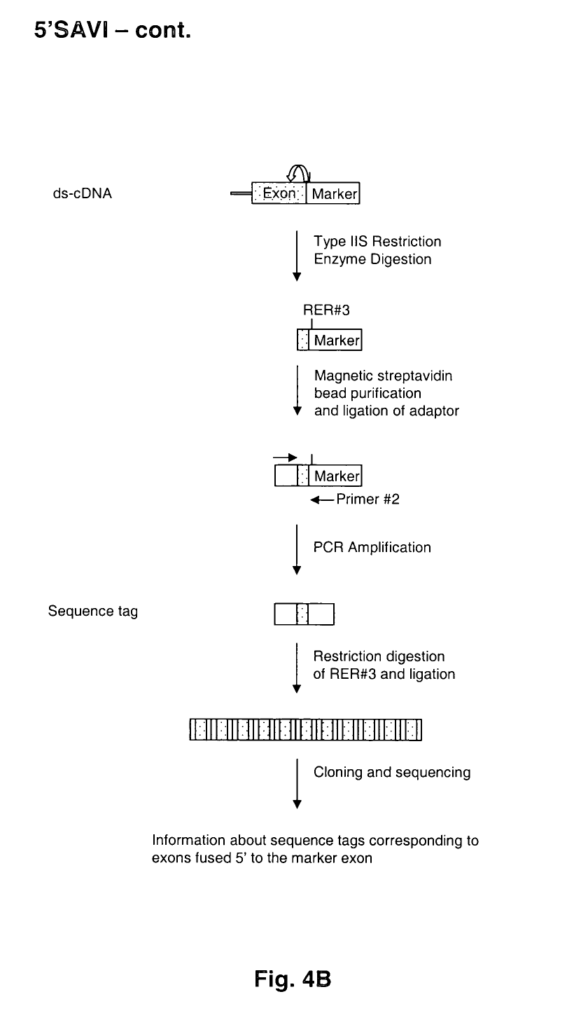

One of these alternative embodiments, denominated 5′ Serial Analysis of Vector Integration (5′SAVI) consists in the identification of sequence tags fused to the 5′ end of the marker exon (FIGS. 4A and 4B). The first step of this method consists in the isolation of spliced mRNA from the cells subjected to random retroviral-mediated integration of the marker exon. Then, a first strand cDNA synthesis is performed with a biotinylated primer complementary to the marker exon region, followed by incubation with a polydeoxynucleotide triphosphate (such as dTTP) and the enzyme Terminal Transferase, to add a homopolymeric tail to the 3′ end of the first cDNA strand. Subsequently, a homopolymeric primer complementary to the homopolymeric tail present on the first strand cDNA is used to prime the synthesis of a second cDNA strand. The end product of this reaction is a population of double stranded cDNAs containing the marker exon fused to the cellular exons located upstream of the marker exon. RNAs that were not tagged by the marker exon do not contribute with sequences to this population of molecules and that greatly reduces the background signals and generation of non-specific sequence tags. The next step consists in the digestion of the double stranded cDNA with a Type IIS restriction enzyme that recognizes RER#1 which will cut upstream of the marker exon, into the cellular exon sequence fused upstream of the 5′ end of the marker exon. The fragments generated by Type IIS restriction enzyme are all of the same size and can be purified by either gel purification or by incubation with magnetic beads bound to streptavidin. The next step of the method consists in the ligation of linkers to the end of the molecule generated by the Type IIS restriction enzyme, followed by PCR amplification with primers complementary to the linker and to the marker exon. After PCR amplification, the fragments are purified, digested with a restriction enzyme that recognizes RER#3, ligated into a concatamer, cloned and sequenced. The process of data aggregation and analysis is similar to what has been described above.

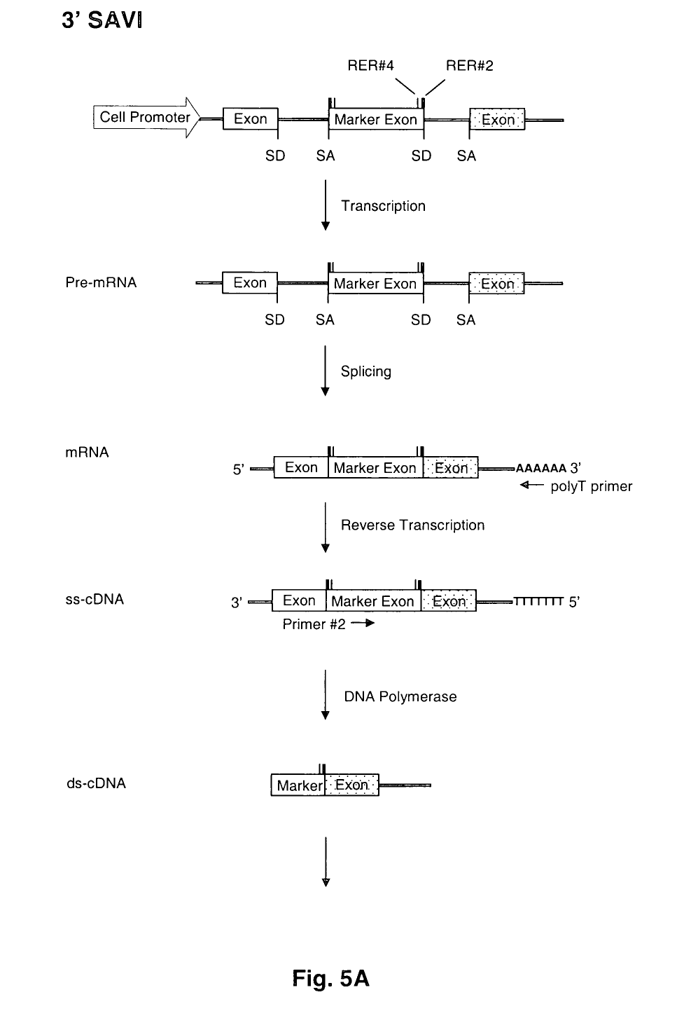

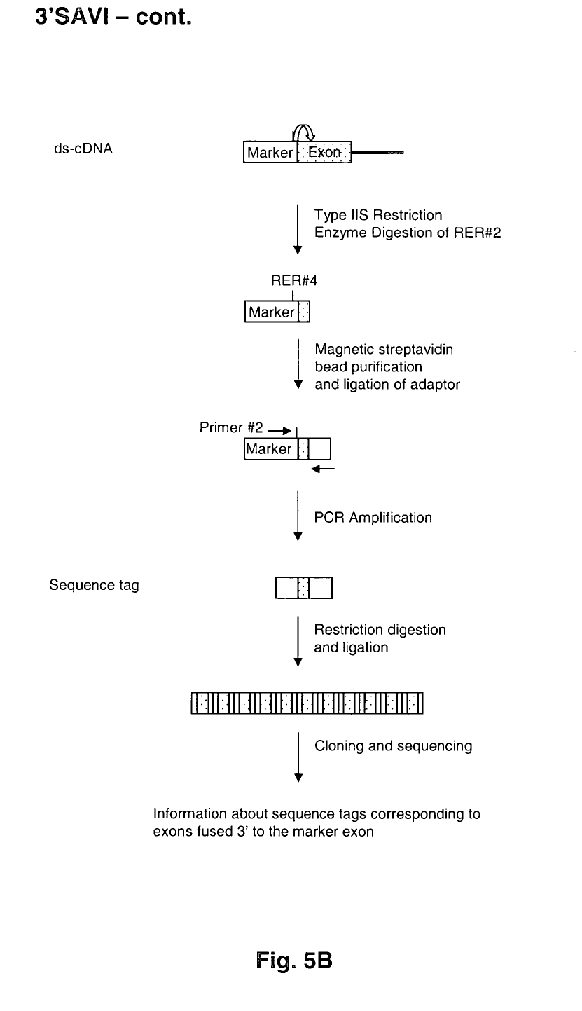

An alternative embodiment of the invention is the method denominated 3′ Serial Analysis of Vector Integration (3′SAVI) (FIGS. 5A and 5B), which consists in the identification of sequence tags of cellular exons fused to the 3′ end of the marker exon. The first step of this method consists in the isolation of spliced mRNA from the cells subjected to random retroviral-mediated integration of the marker exon. Then, a first strand cDNA synthesis is performed with a poly-dT primer complementary to the polyadenylated tail of mRNAs. This reaction is followed by the synthesis of a second cDNA strand with DNA polymerase and a primer corresponding to the plus RNA strand of the marker exon region. The end product of this reaction is a population of double stranded cDNAs containing the marker exon fused to the cellular exons located downstream of the marker exon. RNAs that were not tagged by the marker exon do not contribute with sequences to this population of molecules and that greatly reduces the background signals and non-specific tags. The next step consists in the digestion of the double stranded cDNA with a Type IIS restriction enzyme that recognizes RER#2, which will cut the cDNA downstream of the 3′ end of the marker exon, into the cellular exon sequence fused downstream of the marker exon. The fragments generated by Type IIS restriction enzyme are all of the same size and can be purified by either gel purification or by incubation with magnetic beads bound to streptavidin if the primer used for the second strand cDNA synthesis was biotinylated. The next step of the method consists in the ligation of linkers to the end of the molecule generated by the Type IIS restriction enzyme, followed by PCR amplification with primers complementary to the linker and to the marker exon. After PCR amplification, the fragments are purified, digested with restriction enzyme that recognize RER#4, ligated into a concatamer, cloned and sequenced. The process of data aggregation and analysis is similar to what has been described above.

The SAVI method captures both cellular sequence tags fused upstream and downstream of the marker exon sequence and therefore provides two 14-20 bp tags that are co-linear in the genome, which greatly facilitates assignment of the sequence tags to particular transcriptional units within the genome. In contrast, the methods of 5′SAVI and 3′SAVI provide only one tag of 14-20 bp in length and therefore assignment of the tag to a unique genomic region may not be possible for all tags. Computer modeling using sequence tags of 20 bp in length corresponding to exon-exon junctions of characterized human RNAs suggest that 90% of the sequence tags can be uniquely assigned to a single individual genomic location in the human genome.

According to the invention, a transcriptional profile can be elucidated for any cell type of interest. The invention is particularly useful for comparing cells from different origins or cells from the same origin subjected to different treatments based upon their transcriptional expression profiles. Comparisons can be made between cells from the same tissue from the same organism, between cells from different tissues from the same organism, and cells from different organisms. For example, elucidation and comparison of the transcriptional profiles for a pre-cancerous and/or malignant cell and for a normal cell can be accomplished according to the invention. These profiles can then be compared in order to characterize the molecular events/cellular mechanisms of tumor development. In another application, a cell line could be transduced with the vectors of the invention in order to incorporate tags into its transcriptome. This cell line could be subsequently treated with drugs, hormones, cytokines, subjected to viral infection or other differential treatments and the effects of these substances or treatments could be investigated at the transcriptional level by comparing the transcriptional profiles of both the treated and untreated cell lines.

Recent initiatives in identification of molecular fingerprints of tumors have been focused on studies of DNA and mRNA levels. These studies indicate that gene expression paths in two tumor samples from the same individual were almost always more similar to each other than either was to any other sample and that tumors could be classified in subtypes distinguished by differences in their gene expression patterns.

According to the invention, a test cell and a reference cell could be obtained from the same patient to get a individual transcriptional profile that can be used to diagnose or treat that patient. For example, when a tumor is excised, often a margin of non-transformed cells is removed as well. RNA profiling can help to ensure that the cells removed all had similar profiles to normal cells rather than the metastatic cells from the same patient.

Comparisons may be made according to the invention from different cancers (e.g., lung, breast, colon, melanoma), different stages of malignant progression from corresponding normal tissue to highly malignant primary site and/or metastatic site, tumors caused by endemic/local agents (e.g., environmental agents (asbestos, infectious agents), tissues surrounding the incipient tumor (e.g., blood cells), extracts from body fluids (e.g., cancer cells of the urinary tract may be shed into urine), and tumors from species other than human.

One example of cell lines that may be used as test cells include human tumor cell lines. For example, human tumor cell lines representing a broad spectrum of human tumors and exhibiting acceptable properties and growth characteristics may be grown according to standard operating procedure for cell line expansion, cryopreservation and characterization. Examples of human cancer cell lines which may be used according to the invention include, but are not limited to: Lung Cancer Human Cell Lines (Non-small cell lung cancer adenocarcinoma cell line, A549); adeno squamous cell carcinoma, NCI-H125; squamous cell carcinoma, SK-MES-1, bronchial-alveolar carcinoma, NCI-M322; large cell Carcinoma, A 427, mucoepidermoid carcinoma, NCI-M292, small cell. lung cancer (SCLC) “Classic”, NCI-M69; SCLC “Variant”, NCI-M82; SCLC “Adherent”, SHP77; colon cancer human cell lines (COLO 205, DLD-1, HCT-15, HT29, LoVo); breast cancer human cell lines, (MCF7 WT, MCF7 ADR, MDA-MB-231, HS 578T); prostate cancer human cell lines (D4 145, LNCaP, PC-3, UMSCP-1); melanoma human cell lines (RPMI-7951, LOX, SK-MEL 2, SK-MEL-5, A 375); renal cancer human cell lines (A 498, A 704, Caki-1, SNI2 C, UO-31); ovarian cancer human cell lines (IGROV-1, OVCAR-3, SK-OV-3, A2780, OVCAR-4, OVCAR-5, OVCAR-8); leukemia human cell lines (Molt-4, RPMI 8336, P388, P388/ADR-Resist CCRF-CEM, CCRF-SB); central nervous system cancer human cell lines (SF 126, SF 295, SNB19, SNB 44, SNB 56, TE 671, 4251); sarcoma human cell lines (A-204, A 673, MS 913T, Ht 1080, Te 85); head and neck squamous cancer human cell lines (UM-SCC-MB,C, UM-SCC-21A, UM-SCC-22B); normal fibroblasts (MRC-5-lung, human, CCD-194Lu-lung, human, IMR-90-lung, human, NIH 3T3-mouse, embryo).

Other cell types which could be used include primary cells derived from normal or cancer tissue specimens such as a tissue specimen obtained from normal and/or cancerous tissue that is disaggregated using dissociating enzymes and single cell suspension that is enriched, purified and characterized using MACS tumor cell reagents.

In yet another embodiment, test and reference cells can be used to develop transcriptional profiles associated with aging such as different stages of ontogenesis, for example RNA profiles of embryonic liver-derived hematopoietic stem cell (HSC) vs. cord blood HSC vs. young adult HSC vs. old age organism-derived HSC.

In yet another embodiment, RNA profiles of cells from patients with neurodegenerative diseases such as Alzheimer's disease and Parkinson's disease may be elucidated.

In yet another embodiment, profiles may be obtained for other age-related conditions such as male pattern baldness.

In yet another embodiment, RNA transcriptional profiles can be obtained from human pathological conditions such as genetic diseases (i.e., inborn errors of metabolism, adenosine deaminase deficiency, cystic fibrosis, Duchene's muscular dystrophy).

In yet another embodiment, RNA transcriptional profiles may be obtained for multifactorial and somatic genetic diseases (hypertension, coronary artery disease, obesity, diabetes mellitus).

In yet still another embodiment, RNA transcriptional profiles may be obtained for other non-genetic diseases or acquired genetic diseases such as AIDS.

In yet still another embodiment, profiles may be obtained for autoimmune disorders (i.e., rheumatoid arthritis, systemic lupus erythematosus, multiple sclerosis, etc.) In yet another embodiment, two cells of the same type may be assayed to identify alternative gene forms, such as polymorphic loci, etc. The combination of ditags from the same gene in a given cell may be assayed to identify alternative splicing as well.

In an optional embodiment, the promoterless polynucleotide construct comprising the marker exon may encode for a marker protein capable of generating a fusion protein with the targeted gene. Preferably, and as indicated herein, the marker exon may encode a protein capable of fluorescing, and detection of the protein can be accomplished by fluorescence activated flow cytometry. Because the polynucleotide construct comprising the marker gene does not comprise a promoter operably linked to the marker, expression of the marker will occur only if the construct and, hence, the marker, is integrated into an actively transcribing region of the cell's genome. If the construct integrates into an intron, then, due to the existence of splice acceptor and donor sites flanking the marker, upon cellular transcription, a mRNA will be produced that encodes a fusion protein that includes the marker peptide fused to peptide sequences encoded by upstream and downstream exons. The construct can additionally comprise an internal ribosome entry site (IRES) prior to the start codon of the marker gene, thus ensuring that it will be expressed whenever RNA from the cellular gene (where integration has occurred) is transported to the cytoplasm in a form that is translatable. Moreover, multiple markers may be included such that one marker protein may be expressed as a fusion and a second marker protein may be expressed from an IRES.

The invention however, does not require the expression of a marker gene that can be translated into a protein or fusion protein, and any marker exon that either encodes for a functional reporter protein or not can be used to determine the transcriptional profile of an homogeneous population of cells.

Cells which express the marker are then sorted and preferably quantified by their level of expression to generate an expression profile for a particular cell type. Sorting or separation of the cells can be by any method which provides for the separation and preferably quantification based upon expression of the marker sequence. This could be by fluorescence activation sorting, mechanical sorting, charge or density etc.

A preferred method of sorting includes the use of flow cytometry. Flow cytometry seeks to utilize complex integration of optic, fluidic, and electronic components to develop fluorescence activated cell sorters (FACS) capable of rapid interrogation of cells containing useful fluorescent marker/s in real time.

Marker which may be sorted by this method include cell surface displayed protein; lipid, lipoprotein, glycolipid, and glycoprotein targets that can be tagged with specific fluorescent compounds using labeled antibodies, direct chemical linkage and/or combination of direct and indirect tagging.

One alternative embodiment contemplated includes the use of high-sensitivity/high-density plate readers to detect chemiluminescent signals (range 1×10⁻¹⁸ M to 1×10⁻²¹ M) or with concomitant decreased sensitivity conventional plate reader technology can be used to measure absorbance of enzyme based chromophores. A method for sorting cells with similar speed to that of conventional FACS may be employed where the electrical charging plates are replaced with high performance electromagnets that allow magnetic based separation. Alternatively, confocal microscopy will allow increased sensitivity but with significant reduction in through put.

In another optional embodiment, the polynucleotide construct comprising the marker exon includes a polynucleotide encoding a negative or positive selection protein for enrichment of the population prior to sorting. Use of the negative or positive selection will remove from the population all cells with no integration of the polynucleotide, for example via antibiotic resistance. This provides for enriched populations of target cells to overcome any relative inefficiency of the gene trapping of genomic control elements. Enrichment of gene trapped cells will include the use of drug selection (ex. neo^{r}, puro^{r}, hygro^{r}, zeo^{r}, HAT^{r} etc.), affinity separations to include but not limited to {Ab/Ag or Ab/hapten, biotin/streptavidin, glutathione S-transferase (GST) fusion proteins, Polyhistamine fusion proteins (Invitrogen), calmodulin-binding peptide tag (Stratagene), c-myc epitope tag (peptide seq. EQKLISEEDL) (Stratagene), FLAG epitope tag (peptide seq. DYKDDDDK) (Stratagene), V5 epitope (Stratagene), the Linx™ technology {phenyldiboronic acid [PDBA] and salicylhydroxamic acid [SHA] } (Invitrogen), adhesion, blocking of adhesion, chemotaxis, block of chemotaxis, etc.}, and/or enrichment by FACS using fluorescent Ab, fluorescent Ag, fluorescent substrates or non-fluorescent substrates that become fluorescent after enzymatic cleavage/activation (A complete listing of common fluorescent probes used for applications disclosed herein can be found in Practical Flow Cytometry, 3^{rd} ed., Shapiro, Wiley-Liss (1994); Handbook of Flow Cytometry Methods, Robinson, Wiley-Liss (1993); Flow Cytometry: A Practical Approach, 2^{nd} ed., Ormerod, IRL Press (1994); Current Protocols in Cytometry, Robinson, John Wiley & Sons (2000).

In a preferred embodiment the assay marker gene is a naturally fluorescent protein fusion product that includes but is not limited to green fluorescent protein (GFP) with FACS separation. Examples of uncloned GFP molecules useful for practice of the invention have been sited in Cormier, M. J., Hori, K., and Anderson, J. M. (1974) Bioluminescence in Coelenterates. Biochim. Biophys. Acta 346:137-164. In cases where fluorescent signal of the tagged fusion proteins are of insufficient magnitude to be useful the cells may be probed again with enzyme labeled fluorescence.

The inventive method allows for the study of the mechanism of alternative splicing and the expression of genes regulated in an alternative splicing manner. The transcriptional levels of genes can also be digitized and represented by the frequency of genes being captured. The product of these captured gene tags can be used as probes to hybridize a DNA microarray for data validation.

In accordance with the present invention there may be employed conventional molecular biology, microbiology, and recombinant DNA techniques within the skill of the art. Such techniques are explained fully in the literature. See, e.g., Maniatis, Fritsch & Sambrook, Molecular Cloning: A Laboratory Manual (1982); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription and Translation (B. D. Hames & S. J. Higgins eds. (1984)); Animal Cell Culture (R. I. Freshney, ed. (1986)); Immobilized Cells And Enzymes (IRL Press, (1986)); B. Perbal, A Practical Guide To Molecular Cloning, (1984).

### EXAMPLES

### Example 1

### Cell Transduction and Selection

MCF7 and HMEC cells (5×10⁷ cells) were transduced with 50 ml of pGT13 (FIG. 2) of 10⁶ cfu/ml (Multiplicity of infection approximately 1). GFP positive cells representing successful gene trapping events were sorted by fluorescence activated cell sorting.

### Example 2

### RNA Purification and Recovery of Tags by Serial Analysis of Viral Integration (SAVI).

mRNA was extracted from 107 cells by using poly-dT magnetic beads and separation column (μMACS mRNA isolation Kit, Militenyi Biotec, Auburn, CA). The first-strand cDNA was synthesized by Superscript II reverse transcriptase (Invitrogen), 1 mM dNTPs, using the poly-dT primer attached to magnetic beads at 42° C. for 1 h. First strand cDNA was purified with DNA purification columns (QIAGEN). A poly-dG tail was added at the 3′ end of this first strand cDNA with terminal deoxynucleotide transferase (TdT) with the supply of dGTP 250 μM at 37° C. for 1 h. The second strand cDNA was synthesized by Taq DNA polymerase after the annealing of OLC15 primer (dC₁₅) to the poly-dG tail of the first strand cDNA to become double-stranded cDNA. This double-stranded cDNA was subjected to BsmFI digestion at 65° C. for 3 h. The free ends generated by BsmFI digestion were filled-in by Klenow enzyme and 1 mM dNTPs for 1 h at 37° C. and then subjected to blunt-end ligation with 400,000 units of T4 DNA ligase (16 h at 16° C.) to generate a circular molecule. The di-tag in this circular molecule was first amplified by inverse PCR with primers SAVI#7 (GCACCGCCTGGAGAAG ACCTACG) (SEQ ID NO:2) and SAVI#8 (GGCGGGGCTCAGGATGTCG) (SEQ ID NO:3). The PCR product was used as a template for a second round of PCR amplification with nested primers SAVI#6 (biotin-GAGCAGCACGAGACCGCCATC) (SEQ ID NO:4) and SAVI#9 (GTTGTTCACCACGC CCTCCAG) (SEQ ID NO:5). The PCR product was then subjected to NcoI digestion to drop off the vector sequence at 5′ end of ditag and then purified by streptavidin-conjugated magnetic beads to separate the digestion drop-off from the di-tags. HindIII digestion on the 3′ end of ditag was used to release the ditags from magnetic beads. Pool of ditags were ligated into concatamers and then cloned into pUC 19 which had been previously digested by NcoI, HindIII or both. Each successful ligation of a concatamer into a pUC 19 vector was isolated by transformation of bacteria. This resulted into one concatamer per bacterial colony. Each bacterial colony was used as a template for PCR amplification of concatamer by primers specific to the flanking sequences of the cloning site of concatamer; in this case, PUC 18F (GCCTCTTCGCTATTACGCCAG) (SEQ ID NO:6) and PUC19R (CGGCTCGTATGTTGTGTGGAAT) (SEQ ID NO:7) were used. PCR product was then subjected to Sanger sequencing after the extra primers were removed by Agencourt PCR cleaning kit. The primer for sequencing reaction was either PUC 18F or PUC 18R. Sequenced tags were compared against RefSeq database using BLASTN.

### Example 3

### Recovery of 5′Exon Tags by the Method of 5′SAVI

The gene expression profile of human mammary epithelial cells (HMEC) and human mammary carcinoma cells (MCF7) was compared by using the 5′SAVI method described in this invention. To perform this experiment, 5×10⁷ HMEC or MCF7 cells were transduced with 50 mL of pGTf0 vector (FIG. 2), which uses the MmeI Type IIS restriction enzyme to create tags of 20 base pairs in length. A primer specific to the reporter exon sequence was annealed to the RNA transcripts for the first strand cDNA synthesis toward to the 5′ end of the transcript. A poly-dT tail was added onto the 3′ end of the nascent first strand cDNA by TdT enzyme reaction and dTTP 250 μM. The second strand cDNA was polymerized by Taq polymerase with an oligo-dA₃₅ primer. After digestion with MmeI (1 U/μg DNA, 2 h at 37° C. ), cDNA was purified with PCR purification columns (QIAGEN) and ligated to an adapter synthesized by annealing two complementary oligo-nucleotide strands, 5′-GGG AAT AAG GGC GAC ACG GAA ATG GTA CCN N-3′ (SEQ ID NO:8) (‘N’ denotes a random nucleotide) and 5′p-GGT ACC ATT TCC GTG TCG CCC TTA TTC CC-3′ (SEQ ID NO:9) under the condition of 95° C. for 10 minutes and then cooling down to room temperature at the rate of 1° C. per second. This adapter contains a KpnI recognition sequence at the 5′ end after the two protruding nucleotides. The ligation of this adapter to the MmeI-digested cDNA (overnight ligation with 400,000 U T4 DNA Ligase), allows us to PCR amplify the exon boundary tag (EBT) of a fixed length by using a pair of primers, one specific to the reporter exon and the other is specific to the adapter sequence. The amplification products were cloned into pUC 18 and sequenced by standard techniques. This 5′SAVI approach produced tags of 20 bp rather than an 18 bp uni-tag of a di-tag since the adapter is designed to contain a combination of two protruding nucleotides at the 3′ end to accommodate the 3′ protruding cohesive ends of MmeI-digested molecules. The sequenced tags were compared against RefSeq database using BLAST and some of the results obtained are shown in Table I. The frequency of each class of sequenced tags is used to indicate the relative gene expression level of the transcript to which that tag corresponds. In this experiment, hypothetical protein 20D7-FC4 and hypothetical protein FLJ 13213 were found transcriptionally more active in MCF7 than in HMEC, in contrast, methionine adenosyltransferase II and betaphosphoserine aminotransferase were found more active in HMEC than in MCF7. Other genes were found almost equal in both HMEC and MCF7, such as calcium regulated heat stable protein 1 (24kD), zinc finger protein 274, heterogeneous nuclear ribonucleoprotein A1 and Kruppel-like factor 5 (intestinal).

TABLE I
Examples of frequency and identity of 5′ exon tags in
HMEC and MCF7 cells

| | Tag | |
|---|---|---|
| | Frequency | |
| Transcript ID | HMEC | MCF7 |
| v-abl Abelson murine leukemia viral oncogene | 2 | 0 |
| homolog 2 (arg, Abelson-related gene) | | |
| Actinin, alpha 4 | 0 | 1 |
| annexin A3 | 1 | 1 |
| ATP synthase, H+ transporting, mitochondrial | 7 | 1 |
| F0 complex, subunit e | | |
| Kruppel-like factor 5 (intestinal) | 14 | 10 |
| calmodulin 2 (phosphorylase kinase, delta) | 7 | 0 |
| adaptor-related protein complex 2, sigma 1 subunit | 4 | 2 |
| COX10 homolog, cytochrome c oxidase assembly | 1 | 1 |
| protein, heme A: farnesyltransferase (yeast) | | |
| C-terminal binding protein 2 | 1 | 1 |
| eukaryotic translation elongation factor 1 gamma | 1 | 1 |
| eukaryotic translation elongation factor 2 | 1 | 0 |
| Ferritin, light polypeptide | 0 | 1 |
| Glycyl-tRNA synthetase | 1 | 1 |
| GATA binding protein 6 | 1 | 0 |
| hepatoma-derived growth factor (high-mobility | 6 | 1 |
| group protein 1-like) | | |
| heterogeneous nuclear ribonucleoprotein A1 | 8 | 6 |
| heterogeneous nuclear ribonucleoprotein U (scaffold | 2 | 5 |
| attachment factor A) | | |
| interferon, gamma-inducible protein 16 | 1 | 0 |
| LIM and SH3 protein 1 | 4 | 3 |
| malic enzyme 1, NADP(+)-dependent, cytosolic | 1 | 0 |
| musashi homolog 1 (Drosophila) | 2 | 1 |
| myosin, heavy polypeptide 9, non-muscle | 6 | 0 |
| myosin, heavy polypeptide 9, non-muscle | 6 | 0 |
| ninjurin 1 | 3 | 0 |
| PRKC, apoptosis, WT1, regulator | 0 | 1 |
| ATP-binding cassette, sub-family B (MDR/TAP), | 1 | 0 |
| member 1 | | |
| ribosomal protein L5 | 0 | 1 |
| ribosomal protein L11 | 2 | 1 |
| ribosomal protein L18 | 6 | 4 |
| ribosomal protein L38 | 3 | 2 |
| ribosomal protein S16 | 1 | 0 |
| restin (Reed-Steinberg cell-expressed intermediate | 1 | 0 |
| filament-associated protein) | | |
| S100 calcium binding protein A11 (calgizzarin) | 0 | 1 |
| splicing factor, arginine/serine-rich 10 (transformer | 1 | 0 |
| 2 homolog, Drosophila) | | |
| Solute carrier family 2 (facilitated glucose | 1 | 0 |
| transporter), member 3 | | |
| vesicle-associated membrane protein 2 | 3 | 2 |
| (synaptobrevin 2) | | |
| transmembrane 4 superfamily member 2 | 0 | 4 |
| myeloid/lymphoid or mixed-lineage leukemia 2 | 2 | 1 |
| far upstream element (FUSE) binding protein 1 | 5 | 0 |
| eukaryotic translation initiation factor 2, subunit | 1 | 1 |
| 2 (beta, 38 kD) | | |
| KIAA0226 gene product | 1 | 0 |
| butyrophilin, subfamily 2, member A2 | 1 | 0 |
| tripartite motif-containing 16 | 1 | 0 |
| zinc finger protein 274 | 43 | 30 |
| splicing factor 3a, subunit 3, 60 kD | 1 | 0 |
| GCN1 general control of amino-acid synthesis | 0 | 1 |
| 1-like 1 (yeast) | | |
| butyrophilin, subfamily 2, member A1 | 2 | 0 |
| hypothetical protein 20D7-FC4 | 0 | 10 |
| calcium regulated heat stable protein 1 (24 kD) | 123 | 123 |
| origin recognition complex, subunit 3-like (yeast) | 1 | 0 |
| SH3-domain binding protein 4 | 2 | 0 |
| DKFZP564A2416 protein | 1 | 1 |
| methionine adenosyltransferase II, beta | 24 | 0 |
| phosphoserine aminotransferase | 24 | 2 |
| cytokine receptor-like factor 3 | 1 | 1 |
| butyrophilin, subfamily 2, member A3 | 1 | 0 |
| hypothetical protein FLJ10709 | 3 | 0 |
| hypothetical protein FLJ11099 | 3 | 7 |
| KIAA1170 protein | 1 | 1 |
| p53-induced protein PIGPC1 | 6 | 1 |
| beta globin region | 4 | 6 |
| hypothetical protein FLJ20403 similar to zinc | 0 | 1 |
| finger protein 326 | | |
| Similar to yeast Upf3, variant B | 0 | 2 |
| hypothetical protein FLJ13213 | 0 | 37 |
| AAA-ATPase TOB3 | 3 | 0 |
| par-6 partitioning defective 6 homolog beta (C. | 1 | 1 |
| elegans) | | |
| Similar to Retinol dehydrogenase type I (RODH I) | 3 | 0 |
| Similar to huntingtin-interacting protein HYPA/ | 2 | 0 |
| FBP11 | | |
| LOC204826 | 2 | 0 |

Having described the invention with reference to various methods, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed methods in any sequence which is effective to meet the objectives there intended, unless the context specifically indicates to the contrary.

What is claimed is:

1. A method for elucidating a RNA transcription profile in a eukaryotic cell comprising:
introducing into a cell a polynucleotide construct comprising a polynucleotide sequence, the sequence comprising an exon marker sequence, the expression of which is obtained only upon integration of the polynucleotide construct into an actively transcribing genome region of the cell, wherein the marker sequence is flanked by a 5′ splice acceptor sequence and a 3′ splice donor sequence, wherein the exon marker sequence in a 5′ to 3′ direction contains:
two restriction enzyme recognition (RER) sites located at the 5′ end of the marker exon, wherein at least one of the RER sites is recognized by a Type IIS restriction enzyme, and wherein this RER site is oriented so that a Type IIS restriction enzyme will cut DNA upstream of the 5′ end of the marker exon; and
two restriction enzyme recognition (RER) sites located at the 3′ end of the marker exon, wherein at least one of the RER sites is recognized by a Type IIS restriction enzyme, and wherein this RER site is oriented so that a Type IIS restriction enzyme will cut the DNA downstream of the 3′ end of the marker exon;
reverse transcribing the isolated mRNA into double stranded cDNA;
subjecting the cDNA to digestion with a first Type IIS restriction enzyme that recognizes one of each of the Type IIS RER sites located at the 5′ and 3′ end of the marker exon and cleaving the cDNA upstream of the 5′ end of the marker exon and downstream of the 3′ end of the marker exon, thereby producing a cDNA fragment comprising the marker exon, and portions of upstream and downstream cellular exon tags;
self-ligating the cDNA fragment, thereby fusing the exon tags in opposing orientations;
amplifying a region of the cDNA fragment containing the exon tags in opposing orientations thereby generating a linear DNA molecule containing the exon tags in opposing orientations flanked by sequences corresponding to the marker exon 5′ and 3′ ends;
subjecting the amplified cDNA fragment to one or more restriction enzymes that recognize the RER sites not previously recognized by the first Type IIS restriction enzymes, thereby generating a linear DNA fragment containing upstream and downstream exon tags fused in an inverted conformation;
cloning the fragments comprising the tags in inverted conformation;
obtaining the nucleotide sequence of the cloned tags; and
comparing the individual sequence tags or pairs of sequence tags to a sequence database such that the RNA transcript corresponding to the sequenced tags is identified.

2. The method of claim 1 further comprising ligating the amplified fragments together to form a concatamer prior to cloning.

3 The method of claim 1, wherein the polynucleotide construct is contained within a vector.

4. The method of claim 3, wherein the vector is a viral vector.

5. The method of claim 4, wherein the viral vector is selected from the group consisting of a retroviral vector, a lentiviral vector and an adeno-associated viral vector.

6. The method of claim 5, wherein the viral vector is a retroviral vector.

7. The method of claim 1, wherein the marker exon marker sequence encodes a fluorescent protein.

8. The method of claim 7, wherein the fluorescent protein is green fluorescent protein.

9. The method of claim 8, wherein the fluorescent protein is detected and measured by fluorescence activated flow cytometry.

10. A method for elucidating a RNA transcription profile in a eukaryotic cell comprising:
introducing into a cell a polynucleotide construct comprising a polynucleotide sequence, the sequence comprising an exon marker sequence, the expression of which is obtained only upon integration of the polynucleotide construct into an actively transcribing genome region of the cell, wherein the marker sequence is flanked by a 5′ splice acceptor sequence and 3′ splice donor sequence, wherein the exon marker sequence comprises in a 5′ to 3′ direction:
two restriction enzyme recognition (RER) sites located at the 5′ end of the marker exon, wherein at least one of the RER sites is recognized by a Type IIS restriction enzyme, and wherein this RER site is oriented so that a Type IIS restriction enzyme will cut the DNA upstream of the 5′ end of the marker exon
two restriction enzyme recognition (RER) sites located at the 3′ end of the marker exon, wherein at least one of the RER sites is recognized by a Type IIS restriction enzyme, and wherein this RER site is oriented so that a Type IIS restriction enzyme will cut DNA downstream of the 3′ end of the marker exon;
reverse transcribing the isolated mRNA into single stranded cDNA using a primer of sequence complementary to the sequence of the marker exon;
extending the 3′ end of the single stranded cDNA with a homopolymeric polydeoxynucleotide sequence using a single deoxynucleotide triphosphate and an enzyme terminal transferase;
synthesizing a second and complementary cDNA using a DNA polymerase and a primer complementary to the homopolymeric sequence;
subjecting the cDNA to a Type IIS restriction enzyme that recognizes one of the Type IIS RER sites located at the 5′ end of the marker exon and cleaving the cDNA upstream of the 5′ end of the marker exon, thereby producing a cDNA fragment comprising the marker exon, and portions of upstream cellular exon tags;
ligating a linker to the cDNA fragment;
amplifying the linker and cDNA fragment with primers complementary to the marker exon and to the ligated linker;
subjecting the amplification products to one or more restriction enzymes that recognize the RER sites not previously recognized by the Type IIS restriction enzymes;
cloning the fragments;
obtaining the nucleotide sequence of the cloned tags; and
comparing the individual sequence tags to a sequence database such that the RNA transcript corresponding to the sequenced tags is identified.

11. The method of claim 10 further comprising ligating the amplified fragments together to form a concatamer prior to cloning.

12. The method of claim 10 where the polynucleotide construct comprising the exon marker sequence comprises in the 5′ to 3′ direction:
two restriction enzyme recognition (RER) sites located at the 5′ end of the marker exon, wherein at least one of the RER sites is recognized by a Type IIS restriction enzyme, and wherein this Type IIS RER site is oriented so that a Type IIS restriction enzyme will cut the DNA upstream the 5′ end of the marker exon.

13. The method of claim 10, wherein the step of introducing comprises inserting into a cell a polynucleotide construct, wherein the construct comprises an exon marker sequence, the expression of which is obtained only upon integration of the polynucleotide construct into an actively transcribing genome region of the cell, wherein the marker sequence is flanked at its 5′ end by a splice acceptor sequence, and wherein the exon marker sequence comprises in a 5′ to 3′ direction:
two restriction enzyme recognition (RER) sites located at the 5′ end of the marker exon, wherein at least one of the RER sites is recognized by a Type IIS restriction enzyme, and wherein this Type IIS RER site is oriented so that a Type IIS restriction enzyme will cut the DNA upstream the 5′ end of the marker exon.

14. The method of claim 11, wherein the step of introducing comprises inserting into a cell a polynucleotide construct, wherein the construct comprises an exon marker sequence, the expression of which is obtained only upon integration of the polynucleotide construct into an actively transcribing genome region of the cell, wherein the marker sequence is flanked at its 5′ end by a splice acceptor sequence, wherein the exon marker sequence comprises in a 5′ to 3′ direction:
two restriction enzyme recognition (RER) sites located at the 5′ end of the marker exon, wherein at least one of the RER sites is recognized by a Type IIS restriction enzyme, and wherein this Type IIS RER site is oriented so that a Type IIS restriction enzyme will cut the DNA upstream the 5′ end of the marker exon.

15. The method of claim 10, wherein the polynucleotide construct is contained within a vector.

16. The method of claim 13, wherein the polynucleotide construct is contained within a vector.

17. The method of claim 14, wherein the polynucleotide construct is contained within a vector.

18. The method of claim 15, wherein the vector is a viral vector.

19. The method of claim 18, wherein the viral vector is selected from the group consisting of a retroviral vector, a lentiviral vector and an adeno-associated viral vector.

20. The method of claim 19, wherein the viral vector is a retroviral vector.

21. The method of claim 10, wherein the marker exon sequence encodes a fluorescent protein.

22. The method of claim 21, wherein the fluorescent protein is a green fluorescent protein.

23. The method of claim 22, wherein the fluorescent protein is detected and measured by fluorescence activated flow cytometry.

24. A method for elucidating a RNA transcription profile in a eukaryotic cell comprising:
introducing into a cell a polynucleotide construct comprising a polynucleotide sequence, the polynucleotide sequence comprising an exon marker sequence, the expression of which is obtained only upon integration of the polynucleotide construct into an actively transcribing genome region of the cell, wherein the marker sequence is flanked at its 5′ end by a splice acceptor sequence and at its 3′ end by a splice donor sequence, wherein the exon marker sequence comprises in a 5′ to 3′ direction:
two restriction enzyme recognition (RER) sites located at the 5′ end of the marker exon, wherein at least one of the RER sites is recognized by a Type IIS restriction enzyme, and wherein this RER site is oriented so that a Type IIS restriction enzyme will cut DNA upstream of the 5′ end of the marker exon; and
two restriction enzyme recognition (RER) sites located at the 3′ end of the marker exon, wherein at least one of the RER sites is recognized by a Type IIS restriction enzyme, and wherein this RER site is oriented so that a Type IIS restriction enzyme will cut DNA downstream of the 3′ end of the marker exon,
reverse transcribing the isolated mRNA into single stranded cDNA;
synthesizing a second complementary strand of cDNA with a DNA polymerase enzyme and a primer whose sequence corresponds to the sequence of the marker exon;
subjecting the cDNA to a Type IIS restriction enzyme that recognizes one of the Type IIS RER sites located at the 3′ end of the marker exon and thereupon cleaves the cDNA downstream of the 3′ end of the marker exon, thereby producing a cDNA fragment comprising the marker exon and portions of downstream cellular exon tags;
ligating a linker to the cDNA fragment comprising the marker exon fused to a downstream flanking cellular exon tag;
amplifying the cellular exon tag with primers complementary to the marker exon and to the ligated linker;
subjecting the amplification products to one or more restriction enzymes that recognize the RER sites not previously recognized by the Type IIS restriction enzymes used;
cloning the fragments;
obtaining the nucleotide sequence of the cloned fragments; and
comparing the individual sequence tags to a sequence database such that the RNA transcript corresponding to the sequenced tags is identified.

25. The method of claim 24 further comprising ligating the amplified fragments together to form a concatamer prior to cloning.

26. The method of claim 25 where the polynucleotide construct comprising the exon marker sequence comprises in the 5′ to 3′ direction:
two restriction enzyme recognition (RER) sites located at the 3′ end of the marker exon, wherein at least one of the RER sites is recognized by a Type IIS restriction enzyme, and wherein this Type IIS RER site is oriented so that a Type IIS restriction enzyme will cut the DNA downstream of the 3′ end of the marker exon.

27. The method of claim 24, wherein the step of introducing comprises inserting into a cell a polynucleotide construct, wherein the construct comprises an exon marker sequence, the expression of which is obtained only upon integration of the polynucleotide construct into an actively transcribing genome region of the cell, wherein the marker sequence is flanked at its 5′ by a splice acceptor sequence and at its 3′ end by a splice donor sequence, wherein the exon marker sequence comprises in a 5′ to 3′ direction:
two restriction enzyme recognition (RER) sites located at the 3′ end of the marker exon, wherein at least one of the RER sites is recognized by a Type IIS restriction enzyme, and wherein this RER site is oriented so that a Type IIS restriction enzyme will cut the DNA downstream of the 3′ end of the marker exon.

28. The method of claim 24, wherein the step of introducing comprises inserting into a cell a polynucleotide construct, wherein the construct comprises an exon marker sequence, the expression of which is obtained only upon integration of the polynucleotide construct into an actively transcribing genome region of the cell, wherein the marker sequence is flanked at its 5′ by a splice acceptor sequence and at its 3′ end by a splice donor sequence, wherein the exon marker sequence comprises in a 5′ to 3′ direction:
two restriction enzyme recognition (RER) sites located at the 3′ end of the marker exon, wherein at least one of the RER sites is recognized by a Type IIS restriction enzyme, and wherein this RER site is oriented so that a Type IIS restriction enzyme will cut the DNA downstream of the 3′ end of the marker exon.

29. The method of claim 24, wherein the polynucleotide construct is contained within a vector.

30. The method of claim 29, wherein the vector is a viral vector.

31. The method of claim 30, wherein the viral vector is selected from the group consisting of a retroviral vector, a lentiviral vector and an adeno-associated viral vector.

32. The method of claim 31, wherein the viral vector is a retroviral vector.

33. The method of claim 24, wherein the exon marker sequence encodes a fluorescent protein.

34. The method of claim 33, wherein the fluorescent protein is green fluorescent protein.

35. The method of claim 34, wherein the fluorescent protein is detected and measured by fluorescence activated flow cytometry.

36. A method for elucidating a RNA transcription profile in a cell comprising:
introducing into the cell a polynucleotide marker fragment, the expression of which is obtained only upon integration of the polynucleotide construct into an actively transcribing genome region of the cell, wherein such polynucleotide comprises in a 5′ to 3′ direction:
two restriction enzyme recognition (RER) sites located at the 5′ end of the fragment, wherein at least one of the RER sites is recognized by a Type IIS restriction enzyme, and wherein this RER site is oriented so that a Type IIS restriction enzyme will cut the DNA upstream of the 5′ end of the fragment; and
two restriction enzyme recognition (RER) sites located at the 3′ end of the fragment, wherein at least one of the RER sites is recognized by a Type IIS restriction enzyme, and wherein this RER site is oriented so that a Type IIS restriction enzyme will cut DNA downstream of the 3′ end of the fragment;
reverse transcribing isolated mRNA into double stranded cDNA;
subjecting the cDNA to digestion with a first Type IIS restriction enzyme that recognizes one of each of the Type IIS RER sites located at the 5′ and 3′ end of the marker fragment and cleaving the cDNA upstream of the 5′ end of the marker fragment and downstream of the 3′ end of the marker fragment, thereby producing a cDNA fragment comprising the marker fragment and portions of upstream and downstream cellular RNA tags;
self-ligating the cDNA fragment into a circular molecule, thereby fusing the two RNA tags in opposing orientations;
amplifying a region of the cDNA fragment containing the tags in opposing orientations, thereby generating a linear DNA molecule containing two tags in opposing orientations flanked by sequences corresponding to the marker fragment 5′ and 3′ ends;
subjecting the amplified cDNA fragment to one or more restriction enzymes that recognize the RER sites not previously recognized by the first Type IIS restriction enzymes, thereby generating a linear DNA fragment containing two upstream and downstream tags fused in an inverted conformation;
cloning the fragments comprising the tags in inverted conformation;
obtaining the nucleotide sequence of the cloned fragments; and
comparing the individual sequence tags or pairs of sequence tags to a sequence database such that the RNA transcript corresponding to the sequenced tags is identified.

37. The method of claim 36 further comprising ligating the amplified fragments together to form a concatamer prior to cloning.

38. A method for elucidating a RNA transcription profile in a cell comprising:
introducing into a cell a polynucleotide marker fragment, the expression of which is obtained only upon integration of the polynucleotide construct into an actively transcribing genome region of the cell, wherein the polynucleotide marker fragment comprises in a 5′ to 3′ direction:
two restriction enzyme recognition (RER) sites located at the 5′ end of the fragment, wherein at least one of the RER sites is recognized by a Type IIS restriction enzyme, and wherein this RER site is oriented so that a Type IIS restriction enzyme will cut the DNA upstream of the 5′ end of the fragment;
two restriction enzyme recognition (RER) sites located at the 3′ end of the fragment, wherein at least one of the RER sites is recognized by a Type IIS restriction enzyme, and wherein this RER site is oriented so that a Type IIS restriction enzyme will cut DNA downstream of the 3′ end of the fragment;
reverse transcribing isolated RNA into single stranded cDNA using a primer complementary to the sequence of the marker fragment;
extending the 3′ end of the single stranded cDNA with a homopolymeric polydeoxynucleotide sequence using a single deoxynucleotide triphosphate and an enzyme terminal transferase;
synthesizing a second and complementary cDNA strand using a DNA polymerase and a primer complementary to the homopolymeric sequence;
subjecting the cDNA to a Type IIS restriction enzyme that recognizes one of the Type IIS RER sites located at the 5′ end of the marker fragment and thereupon cleaving the cDNA upstream of the 5′ end of the marker fragment, thereby producing a cDNA fragment comprising the marker fragment and portions of upstream cellular tags;
ligating a linker to the cDNA fragment;
amplifying the ligation products with primers complementary to the marker fragment and to the ligated linker;
subjecting the amplification products to one or more restriction enzymes that recognize the RER sites not previously recognized by the Type IIS restriction enzymes used;
cloning the fragments;
obtaining the nucleotide sequence of the cloned fragments; and
comparing the individual sequence tags to a sequence database such that the RNA transcript corresponding to the sequenced tags is identified.

39. The method of claim 38 further comprising ligating the amplified fragments together to form a concatamer prior to cloning.

40. A method for elucidating a RNA transcription profile in a cell comprising:
introducing into the cell a polynucleotide marker fragment, the expression of which is obtained only upon integration of the polynucleotide construct into an actively transcribing genome region of the cell, wherein the polynucleotide marker fragment comprises in a 5′ to 3′ direction:
two restriction enzyme recognition (RER) sites located at the 5′ end of the marker fragment, wherein at least one of those RER sites is recognized by a Type IIS restriction enzyme, and wherein this RER site is oriented so that a Type IIS restriction enzyme will cut the DNA upstream of the 5′ end of the fragment;
two restriction enzyme recognition (RER) sites located at the 3′ end of the marker fragment, wherein at least one of the RER sites is recognized by a Type IIS restriction enzyme, and wherein this RER site is oriented so that a Type IIS restriction enzyme will cut DNA downstream of the 3′ end of the marker fragment;
reverse transcribing isolated RNA into single stranded cDNA;
synthesizing a second complementary strand of cDNA with a DNA polymerase and a primer whose sequence corresponds to the sequence of the marker fragment;
subjecting the cDNA to a Type IIS restriction enzyme that recognizes one of the Type IIS RER sites located at the 3′ end of the marker fragment and cleaving the cDNA downstream of the 3′ end of the marker fragment, such that a cDNA fragment is produced comprising the marker, and portions of downstream cellular tags; ligating a linker to the cDNA fragment;
amplifying the ligation products with primers corresponding to the marker and to the ligated linker;
subjecting the amplification products to one or more restriction enzymes that recognize the RER sites not previously recognized by the Type IIS restriction enzymes used;
cloning the fragments;
obtaining the nucleotide sequence of the cloned fragments; and
comparing the individual sequence tags to a sequence database such that the RNA transcript corresponding to the sequenced tags is identified.

41. The method of claim 40 further comprising ligating the amplified fragments together to form a concatamer prior to cloning.

42. The method of claim 36, wherein the polynucleotide marker fragment is introduced into the cell by transfection.

43. The method of claim 37, wherein the polynucleotide marker fragment is introduced into the cell by transfection.

44. The method of claim 38, wherein the polynucleotide marker fragment is introduced into the cell by transfection.

45. The method of claim 39, wherein the polynucleotide marker fragment is introduced into the cell by transfection.

46. The method of claim 40, wherein the polynucleotide marker fragment is introduced into the cell by transfection.

47. The method of claim 41, wherein the polynucleotide marker fragment is introduced into the cell by transfection.

48. A polynucleotide construct comprising in a 5′ to 3′ orientation:
a splice acceptor sequence;
a Type IIS restriction enzyme recognition site oriented so that it is capable of cleaving DNA fused upstream of the 5′ end of a marker exon;
a restriction enzyme recognition site;
a marker exon;
a restriction enzyme recognition site;
a Type IIS restriction enzyme recognition site oriented so that it is capable of cleaving sequences located downstream of the 3′ end of the marker exon; and
a splice donor sequence.

49. A vector comprising the polynucleotide construct of claim 48.

50. The polynucleotide construct of claim 48 further comprising a polynucleotide sequence encoding a positive selection marker located downstream from the marker exon.

51. The polynucleotide construct of claim 48, wherein the marker exon encodes a fluorescent protein marker.

52. The polynucleotide construct of claim 51, wherein the fluorescent protein marker is green fluorescent protein.

53. The polynucleotide construct of claim 48, wherein the Type IIS restriction enzyme recognition site is selected from the group consisting of BsmFI and MmeI.

54. The polynucleotide construct of claim 48, wherein the restriction enzyme recognition site is selected from the group consisting NcoI and BamHI.

55. A polynucleotide construct comprising in a 5′ to 3′ orientation:
a splice acceptor sequence;
a restriction enzyme recognition site;
a Type IIS restriction enzyme recognition site oriented so that it is capable of cleaving DNA fused upstream of the 5′ end of a marker exon;
a marker exon;
a Type IIS restriction enzyme recognition site oriented so that it is capable of cleaving sequences located downstream of the 3′ end of the marker exon;
a restriction enzyme recognition site;
a splice donor sequence.

56. A vector comprising the polynucleotide construct of claim 55.

57. The polynucleotide construct of claim 55 further comprising a polynucleotide sequence encoding a positive selection marker located downstream from the marker exon.

58. The polynucleotide construct of claim 55, wherein the marker exon encodes a fluorescent protein marker.

59. The polynucleotide construct of claim 58, wherein the fluorescent protein marker is green fluorescent protein.

60. The polynucleotide construct of claim 55, wherein the Type IIS restriction enzyme recognition site is selected from the group consisting of BsmFI and MmeI.

61. The polynucleotide construct of claim 55, wherein the restriction enzyme recognition site is selected from the group consisting NcoI and BamHI.

62. A polynucleotide construct comprising in a 5′ to 3′ orientation:
a splice acceptor sequence;
a Type IIS restriction enzyme recognition site oriented so that it is capable of cleaving DNA fused upstream of the 5′ end of a marker exon;
a restriction enzyme recognition site; and
a marker exon; and
a polyadenylation sequence.

63. A vector comprising the polynucleotide construct of claim 62.

64. The polynucleotide construct of claim 62, wherein the marker exon encodes a fluorescent protein marker.

65. The polynucleotide construct of claim 64, wherein the fluorescent protein marker is green fluorescent protein.

66. The polynucleotide construct of claim 62, wherein the Type IIS restriction enzyme recognition site is MmeI or BsmFI.

67. The polynucleotide construct of claim 62, wherein the restriction enzyme recognition site is NheI.

68. A polynucleotide construct comprising:
a marker exon sequence flanked by a functional 5′ splice acceptor sequence and a 3′ splice donor sequence, and
wherein said marker exon contains at least two restriction enzyme recognition (RER) sites at the 5′ end of the marker exon, wherein at least one the 5′ RER sites is recognized by a Type IIS restriction enzyme and oriented in such a way that a Type IIS restriction enzyme cuts the DNA outside the boundaries that define the marker exon, and
wherein the marker exon contains at least two RER sites at the 3′ end of the marker exon, wherein at least one of the 3′ RER sites is recognized by a Type IIS restriction enzyme and oriented in such a way that a Type IIS restriction enzyme cuts the DNA outside the boundaries that define the marker exon, and
wherein said restriction recognition sites are located close from the border of the marker exon such that after cutting flanking exons generate sequence tags of at least 8 nucleotides.

69. A vector comprising the polynucleotide construct of claim 68.

70. A polynucleotide construct comprising:
a marker exon sequence flanked by a functional 5′ splice acceptor sequence and a 3′ splice donor sequence, and
wherein said marker exon contains at least two restriction enzyme recognition (RER) sites at the 5′ end of the marker exon, wherein at least one of the RER sites is recognized by a Type IIS restriction enzyme, and
wherein this Type IIS RER site is oriented so that a Type IIS restriction enzyme will cut DNA upstream of the 5′ end the marker exon.

71. A vector comprising the polynucleotide construct of claim 70.

72. A polynucleotide construct comprising:
a marker exon sequence flanked by a functional 5′ splice acceptor sequence and a 3′ splice donor sequence, and
wherein said marker exon contains at least two restriction enzyme recognition (RER) sites at the 3′ end of the marker exon, wherein at least one of the RER sites is recognized by a Type IIS restriction enzyme, and
wherein this Type IIS RER site is oriented so that a Type IIS restriction enzyme will cut DNA downstream of the 3′ end the marker exon.

73. A vector comprising the polynucleotide construct of claim 72.

74. A polynucleotide construct pGT13.

75. A polynucleotide construct pGTfso-M.
